(19) United States
(12) Patent Application Publication
(10) Pub. No.: US 2012/0203004 A1
(43) Pub. Date: Aug. 9, 2012

(54) PROCESS FOR THE SYNTHESIS OF ALKYL/ARALKYL (2S)-2-(TERT-BUTOXYCARBONYL)-AMINO-2-[-8-AZABICYCLO[3.2.1]OCT-3-YL]-EXO-ACETATE AND ANALOGS THEREOF: KEY INTERMEDIATES FOR THE PREPARATION OF DPPIV INHIBITORS

(75) Inventors: Bhairab Nath Roy, Maharashtra (IN); Rajender Kumar Kamboj, Maharashtra (IN); Shaji K. Goerge, Maharashtra (IN); Spinvin C. Venugopal, Maharashtra (IN); Muthu Kumaran Shanmugvadivelu, Maharashtra (IN); Neelima Sinha, Maharashtra (IN)

(73) Assignee: LUPIN LIMITED, Mumbai - 400 098, Maharashtra (IN)

(21) Appl. No.: 13/390,157

(22) PCT Filed: Apr. 28, 2010
(86) PCT No.: PCT/IN2010/000276

§ 371 Date: Apr. 23, 2012

(30) Foreign Application Priority Data
Aug. 13, 2009 (IN) 1055/KOL/2009

(51) Int. Cl. C07D 451/02

(52) U.S. Cl. 546/132; 546/124

(57) ABSTRACT

An improved process for the synthesis of intermediates like Alkyl/Aralkyl (2S)-2-(tert-butoxycarbonyl)-amino-2-[-8-azabicyclo[3.2.1]oct-3-yl]-exo-acetate and analogs thereof which are useful in the synthesis of Dipeptidyl peptidase-IV (DP-PIV) inhibitors.

### FIELD OF INVENTION

The present invention provides an improved process for the synthesis of various intermediates useful in the synthesis of DPPIV inhibitors, as described in WO 2009/037719 A1.

### BACKGROUND OF THE INVENTION

Dipeptidyl peptidase-IV (DPP-IV) is a membrane bound serine protease, which is widely expressed in mammalian tissues such as intestine, liver, lung, kidney etc. as a type II integral membrane protein. The substrate for DPP-IV includes chemokines, neuropeptides, vasoactive peptides, GLP-1, GLP-2, GHRH and NPY. Recent studies have shown that inhibition of DPP-IV increases the level of circulating GLP-1 and thus increases the insulin secretion, an important property in developing therapeutics for the treatment of Type II diabetes. (Ahren, B et al, Eur. J. Pharmacol. 2000, 404, 239; Paul E. Wiedeman, Progress in Medicinal Chemistry 2007, 45, 63; Peter Kirkpatrick, Nature Reviews Drug Discovery 2, 92, February 2003).

Synthesis of a series of novel and potent DPPIV inhibitor(s) using a key intermediate, methyl (2S)-2-(tert-butoxycarbonyl)-amino-2-[-8-azabicyclo[3.2.1]oct-3-yl]-exo-acetate and its analogs (compounds of formula m, n and o, Scheme 1) is described in WO 2009/037719 A1 and PCT/IN2009/000478. The methodology disclosed in WO 2009/037719 A1 and PCT/1N2009/000478, which is described below in scheme 1, employed the use of metal cyanides under acidic condition as well as Zn powder, in large excess. In addition to the above, the starting material, 3-hydroxymethyl-8-methyl-8-aza-bicyclo[3.2.1]octan-3-ol (compound of formula a, Scheme 1) is prepared from tropinone by a four-step conversion involving the use of sodium cyanide in aqueous hydrochloric acid and Lithium aluminium hydride as well. Neither these chemicals are safe to handle nor are they eco-friendly on a larger scales. Further, due to large number of steps involved (17 steps), the overall yield has been found to be less than 10% and thereby very poor atom economy.

Because of the various reasons mentioned hereinbefore, attempts to prepare methyl (2S)-2-(tert-butoxycarbonyl)-amino-2-[-8-azabicyclo[3.2.1]oct-3-yl]-exo-acetate (compound n) on a larger scale is not practical and hence an alternative chemistry, which incorporates superior safety, atom-economy and hazard-free processes needs to be developed. The present invention provides a solution for most of the problems existing in the reported method for the synthesis of compound of formula n and its analogues of formula m and o.

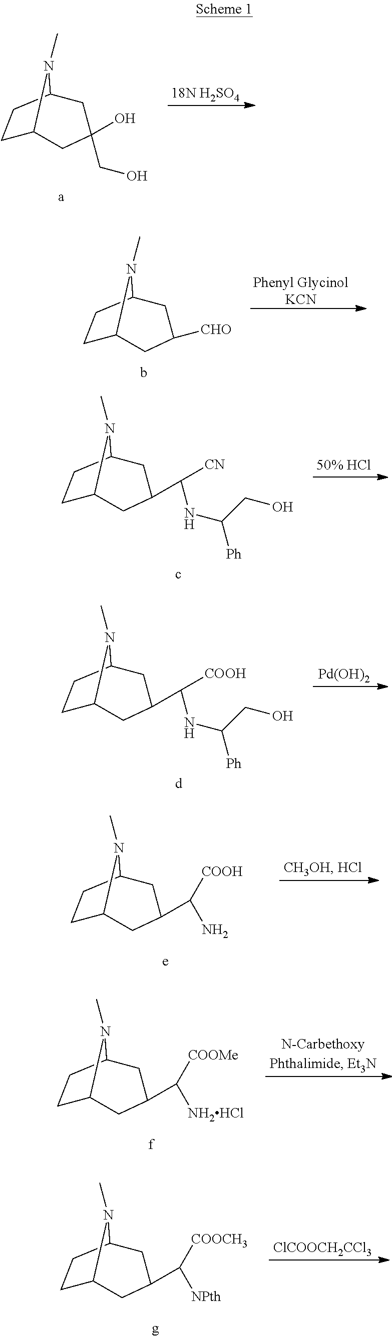

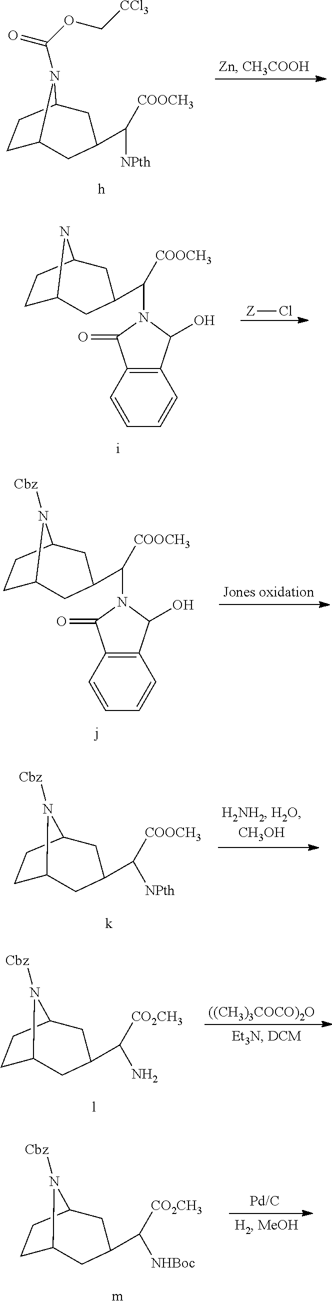

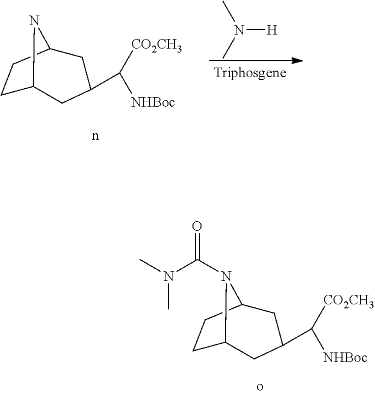

### SUMMARY OF INVENTION

The present invention relates to an improved process for the preparation of a key intermediate viz. methyl (2S)-2-(tert-butoxycarbonyl)-amino-2-[-8-azabicyclo[3.2.1]oct-3-yl]-exo-acetate (compound of formula n) useful for the preparation of DPP-IV inhibitors, as schematically provided in ‘Scheme-A’.

Scheme A illustrates various approaches towards the syntheses of compound of the formula n and its analogues in comparison with the process reported in WO 2009/037719 A1 and PCT/IN2009/000478. The process reported in the cited literature employed 17 steps (Scheme 1) against which the present strategies as outlined in scheme 2, 3 and 4 offers more practical, cost effective, safe and eco-friendly process routes. Scheme 2 leads to the intermediate n in 8 steps and o in 9 steps, scheme 3 offers o in 8 steps, and scheme 4 offers n in 6 steps and o in 7 steps.

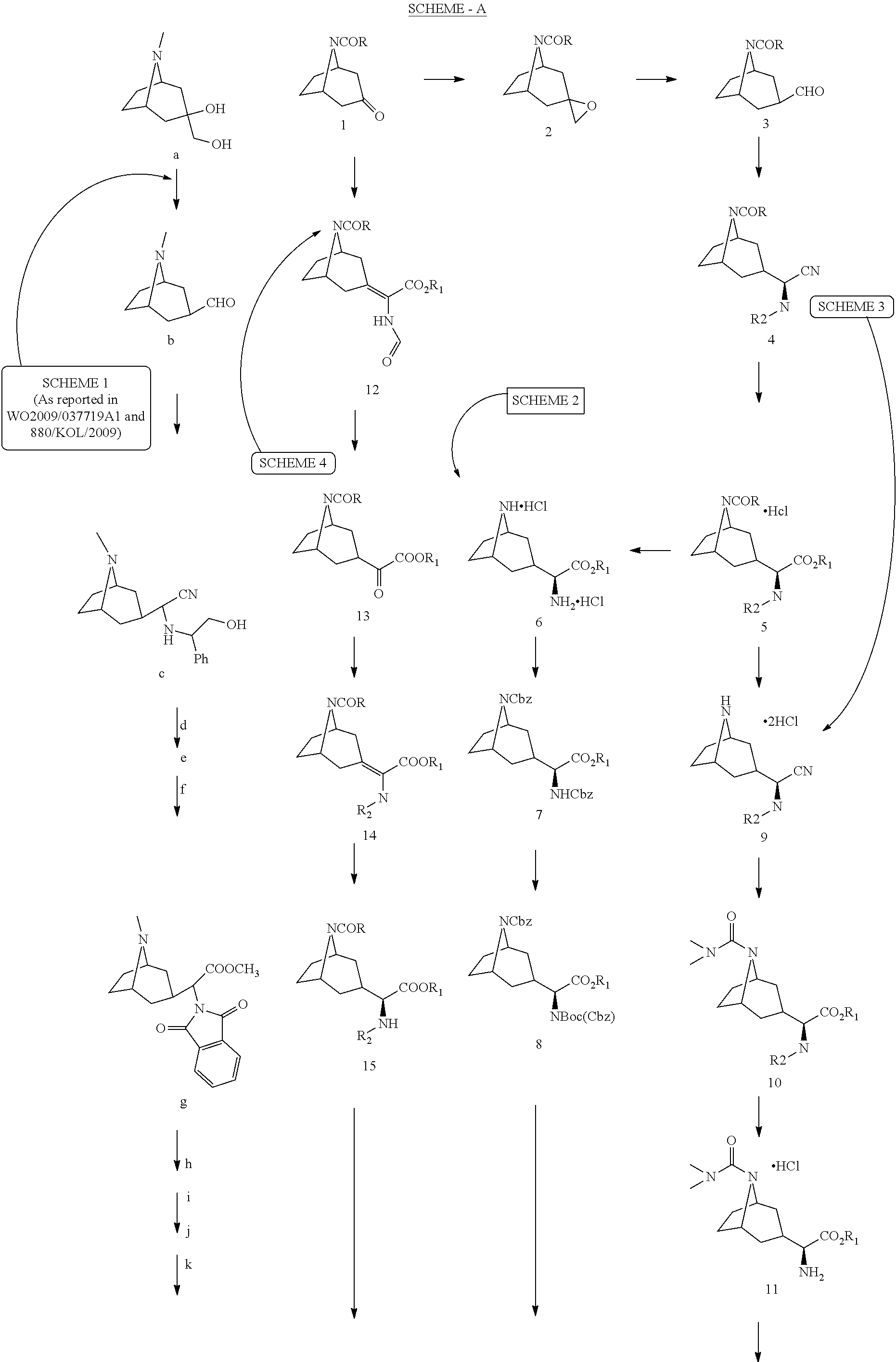

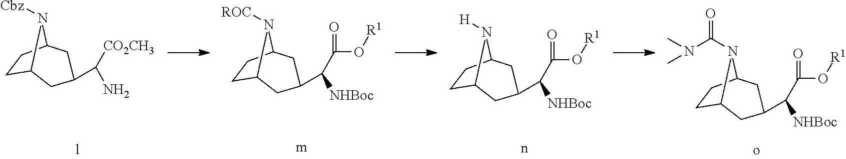

As mentioned hereinabove the present invention provides an alternative improved processes for the synthesis of methyl (2S)-2-(tert-butoxycarbonyl)-amino-2-[-8-azabicyclo[3.2.1]oct-3-yl]-exo-acetate (compound of formula n) and its analogue comprising of:
Sequence as depicted in scheme 2.
Sequence as depicted in scheme 3.
Sequence as depicted in scheme 4.

### Scheme 2:

(i) conversion of benzyl 3-oxo-8-azabicyclo[3.2.1]octane-8-carboxylate (compound 1) to benzyl 3-exo-formyl-8-azabicyclo[3.2.1]octane-8-carboxylate (compound 3) using two-step procedure employing trimethylsulfonium iodide ((CH₃)₃S⁺I) and boron trifluoride-diethyl etherate (BF₃-etherate) in dichloro methane (DCM).

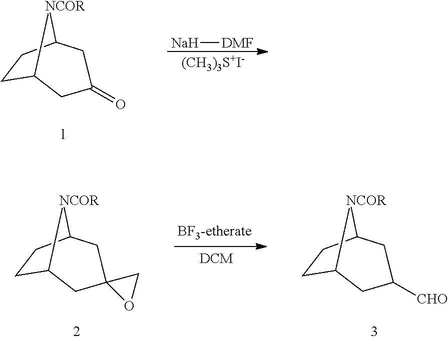

(ii) conversion of benzyl 3-exo-formyl-8-azabicyclo[3.2.1]octane-8-carboxylate (compound 3) to 1-(2-hydroxy-1-(1R)-phenylethyl amino)-1-(8-(benzyloxy carbonyl)-8-aza-bicyclo[3.2.1]-oct-3-yl)-exo-methane-1-(15)-carbonitrile (compound 4) following the Strecker synthesis using R-(−)-2-phenyl glycinol as the chiral auxiliary and trimethylsilyl cyanide (TMSCN) as the cyanide source, and the reaction can be carried out in either alcoholic or chlorinated solvents in general, methanol (MeOH) or dichloromethane (DCM) or chloroform (CHCl₃) in particular in the presence of acetic acid (AcOH) or any other Lewis acid.

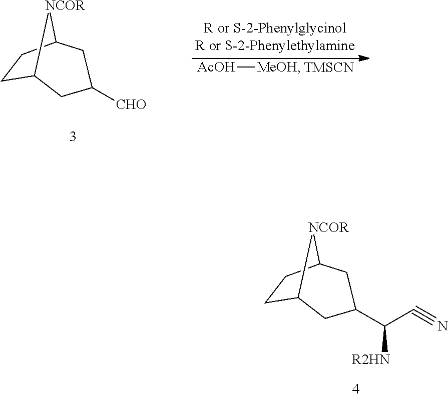

(iii) hydrolysis of the nitrile group in 1-(2-hydroxy-1-(1R)-phenylethyl amino)-1-(8-(benzyloxycarbonyl)-8-aza-bicyclo[3.2.1]-oct-3-yl)-exo-methane-1-(1S)-carbo-nitrile (compound 4) using methanolic-hydrogen chloride (MeOH—HCl) to obtain 1-(2-hydroxy-1-(1R)-phenylethylamino)-1-(8-(benzyloxycarbonyl)-8-aza-bicyclo-[3.2.1]-oct-3-yl)-exo-methane-1-(1S)-carboxylic acid methyl ester hydrochloride (compound 5).

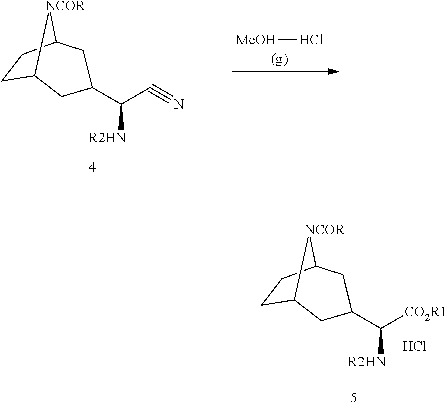

(iv) conversion of 1-(2-hydroxy-1-(1R)-phenylethyl amino)-1-(8-(benzyloxy carbonyl)-8-aza-bicyclo[3.2.1]-oct-3-yl)-exo-methane-1-(1S)-carboxylic acid methyl ester hydrochloride (compound 5) to methyl (25)-2-amino-2-[8-azabicyclo[3.2.1]oct-3-yl]-exo-acetate to hydrochloride (compound 6) by the de-protection of both amino groups using an elevated pressure of hydrogen over palladium supported on charcoal (Pd/C).

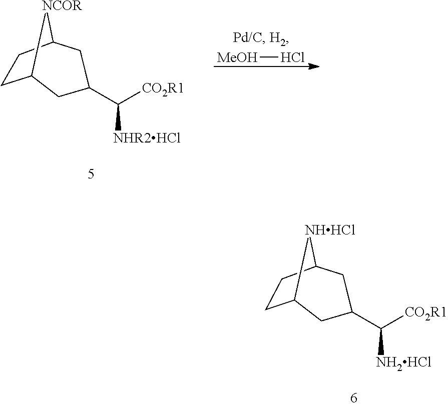

(v) protection of the both amino groups in methyl (2S)-2-amino-2-[8-azabicyclo-[3.2.1]oct-3-yl]-exo-acetate hydrochloride (compound 6) using benzyl chloroformate (ZCl) in the presence of aqueous saturated sodium bicarbonate solution (aq. NaHCO₃) to give methyl (2S)-2-(benzyloxycarbonyl)-amino-2-[-8-[benzyloxy carbonyl]-8-azabicyclo-[3.2.1]oct-3-yl]-exo-acetate (compound 7).

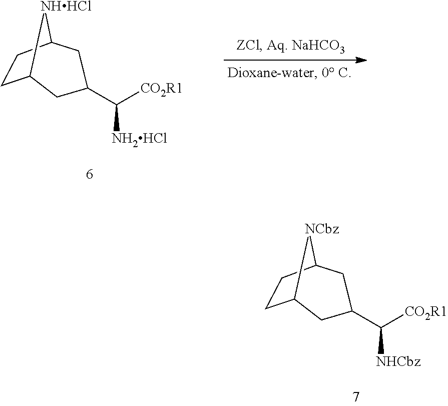

(vi) conversion of methyl (2S)-2-(benzyloxycarbonyl)-amino-2-[-8-benzyloxy carbonyl]-8-azabicyclo[3.2.1]oct-3-yl]-exo-acetate (compound 7) to methyl (2S)-2-((tert-butoxycarbonyl)-[benzyloxy carbonyl]-amino)-2-[8-(benzyloxy carbonyl)-8-azabicyclo[3.2.1]oct-3-yl]-exo-acetate (compound 8) using di-tert-butyl dicarbonate ([(CH3)3COCO]2O) and 4-dimethylaminopyridine (DMAP) in an aprotic organic solvent such as acetonitrile (ACN).

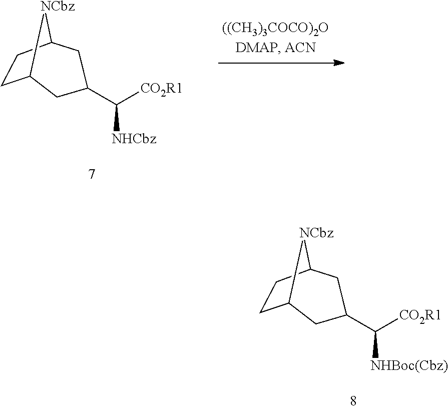

(vii) hydrogenolysis of methyl (2S)-2-((tert-butoxycarbonyl)-[benzyloxy carbonyl]-amino)-2-[8-(benzyloxycarbonyl)-8-azabicyclo[3.2.1]oct-3-yl]-exo-acetate (compound 8) to over palladium supported on charcoal (Pd/C) to give methyl (2S)-2-(tert-butoxycarbonyl)-amino-2-[-8-azabicyclo[3.2.1]oct-3-yl]-exo-acetate (compound n). Compound n can be further converted to compound o on treatment with dimethyl amine and triphosgene, or N,N-dimethylcabamoyl chloride in an appropriate solvent in presence of base like triethyl amine, diisopropyl ethyl amine etc.

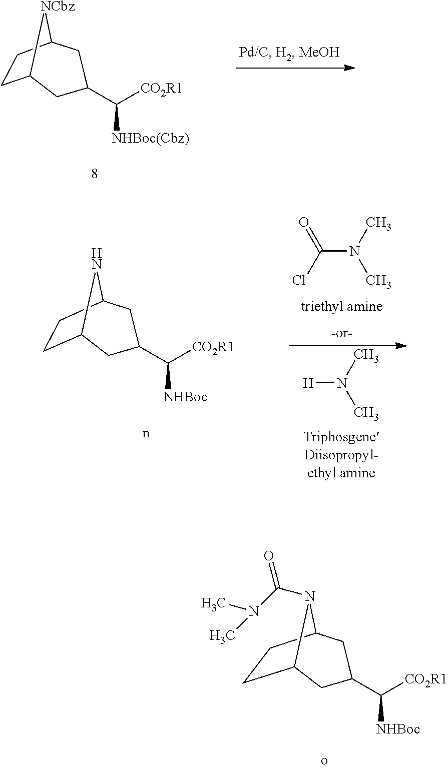

### Scheme 3:

Another aspect of the present invention is to provide an improved process as shown in scheme 3, vide infra, for the synthesis of compound of formula o which is an advanced key intermediate for the synthesis of DPPIV inhibitor analogues derived from the above strategy comprising of:

i) conversion of 1-(2-hydroxy-1-(1R)-phenylethyl amino)-1-(8-(benzyloxy carbonyl)-8-aza-bicyclo[3.2.1]-oct-3-yl)-exo-methane-1-(1S)-carbonitrile hydrochloride salt (compound 4) to 1-(2-hydroxy-1-(1R)-phenylethyl amino)-1-(8-(dimethyl carbamoyl)-8-aza-bicyclo[3.2.1]-oct-3-yl)-exo-methane-1-(1S)-carboxylic acid methyl ester (compound 10) by treating a solution of compound 4 with dry hydrogen chloride gas for extended period of time at room temperature to give (S)-(8-Aza-bicyclo[3.2.1]oct-3-yl)-((R)-2-hydroxy-1-phenyl-ethylamino)-acetic acid methyl ester 9 followed by regio-selective introduction of N,N-dimethyl carbamoyl group using N,N-dimethyl carbamoyl chloride or N,N-dimethylamine and triphosgene on the bridge head nitrogen in the presence of an organic base such as diisopropylethylamine or triethylamine or using an inorganic bases such as cesium carbonate in an appropriate organic solvents such as dimethyl formamide or dimethyl acetamide or in any water miscible cyclic ether.

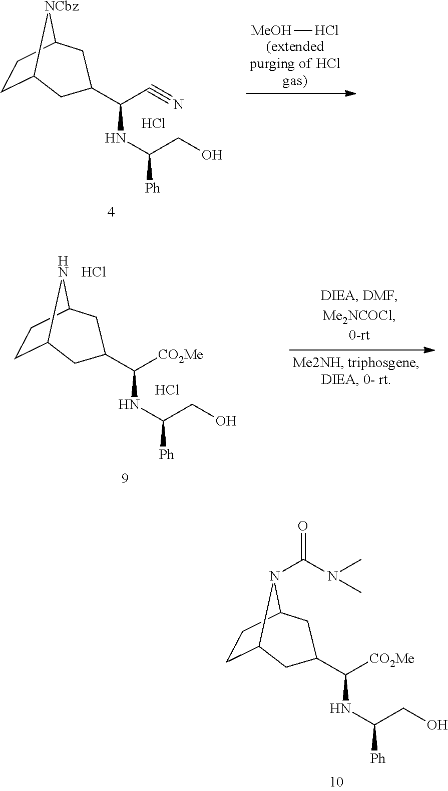

### —OR—

conversion of 1-(2-hydroxy-1-(1R)-phenylethyl amino)-1-(8-(benzyloxy carbonyl)-8-aza-bicyclo[3.2.1]-oct-3-yl)-exo-methane-1-(15)-carboxylic acid methyl ester hydrochloride salt (compound 5) to 1-(2-hydroxy-1-(1R)-phenylethyl amino)-1-(8-(dimethyl carbamoyl)-8-aza-bicyclo[3.2.1]-oct-3-yl)-exo-methane-1-(1S)-carboxylic acid methyl ester (compound 10) by selective hydrogenation of benzyloxy carbonyl group in 5 to give (S)-(8-Aza-bicyclo[3.2.1]oct-3-yl)-((R)-2-hydroxy-1-phenyl-ethylamino)-acetic acid methyl ester 9 using Pd/C catalyst at an elevated pressure of hydrogen at room temperature followed by regio-selective introduction of N,N-dimethyl carbamoyl group using N,N-dimethyl carbamoyl chloride or N,N-dimethylamine and triphosgene on the bridge head nitrogen in the presence of an organic base such as diisopropylethylamine or triethylamine or using an inorganic bases such as cesium carbonate in an appropriate organic solvents such as dimethyl formamide or dimethyl acetamide or in any water miscible cyclic ether.

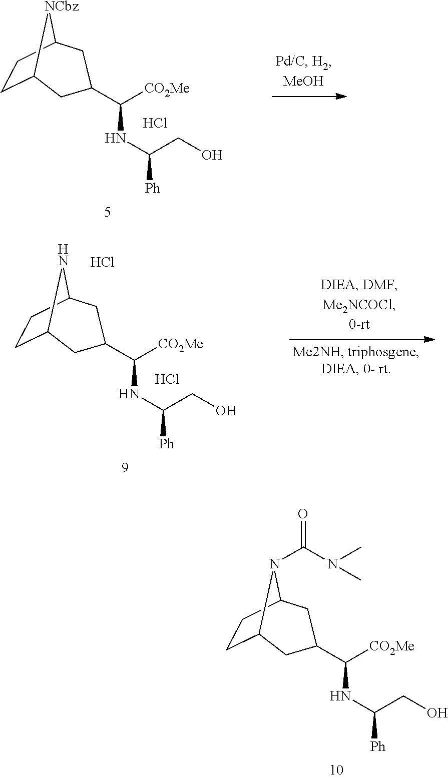

ii) conversion of 1-(2-hydroxy-1-(1R)-phenylethyl amino)-1-(8-(dimethyl carbamoyl)-8-aza-bicyclo[3.2.1]-oct-3-yl)-exo-methane-1-(1S)-carboxylic acid methyl ester (compound 10) to methyl (2S)-2-(tert-butoxycarbonyl)-amino-2-[-8-[dimethylcarbamoyl]-8-azabicyclo[3.2.1]-oct-3-yl]-exo-acetate (compound o) in two steps comprising of removal of the phenyl ethanol group in 10 by hydrogenation in presence of Pd/C catalyst and acetic acid in an organic solvent such as methanol at around 20 Kg hydrogen pressure to form compound of formula 11, followed by protection of the free amine by treatment with di-tert-butyl dicarbonate ([(CH₃)₃COCO]₂O) in dichloromethane.

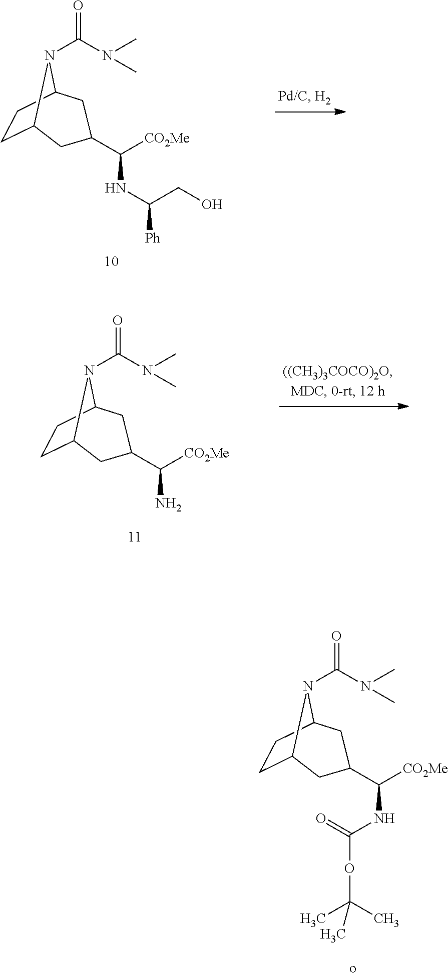

### Scheme 4:

Yet another aspect of the present invention is to provide an improved process as shown in scheme 4, vide infra, for synthesis of compound of formula o via compounds m and n.

conversion of benzyl 3-oxo-8-azabicyclo[3.2.1]octane-8-carboxylate (compound 1) to benzyl 3-(1-formamido-2-ethoxy-2-(oxo)ethyl idene)-8-azabicyclo[3.2.1]-octane-8-carboxylate (compound 12) by the condensation of compound of formula 1 with an active methylene compounds such as methyl or ethyl isocyanoacetate in the presence of sodium hydride (NaH) in an aprotic solvent such as tetrahydrofuran (THF) or in 1,4-dioxane or in any water miscible cyclic ether.

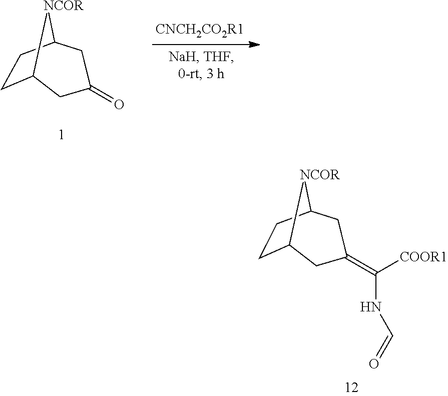

i. conversion of compound of formula 12 to a mixture of benzyl exo-3-[ethoxy or methoxy (oxo)acetyl]-8-azabicyclo[3.2.1]octane-8-carboxylate (compound 13) by treatment with methanolic hydrogen chloride (MeOH—HCl).

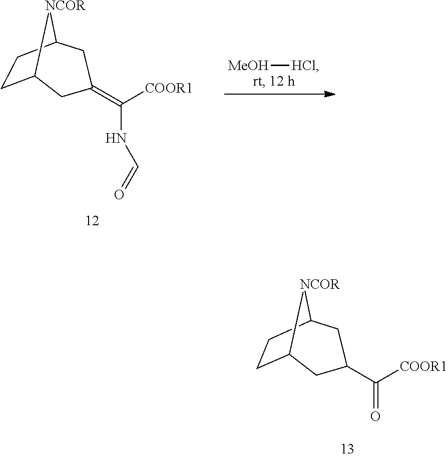

ii. conversion of compound of formula 13 to 1-((either S or R)-tert-butylsulfinylamino)-1-(8-(benzyloxycarbonyl)-8-aza-bicyclo[3.2.1]-oct-3-yl)-exo-methane-1-(1S)-carboxylic acid ethyl ester (compound 15) by first treating compound 13 with either (S)-(+2-methyl-2-propane sulfinamide or with (R)-(+)-2-methyl-2-propane sulfinamide in the presence of titanium(IV) ethoxide (Ti(OEt)₄) and followed by further reduction of the intermediate ketimine 14, using a suitable reducing agent such as sodium borohydride (NaBH₄) or sodium triacetoxy borohydride ((CH₃COO)₃BHNa) or any chiral or achiral reducing agents derived from a combination of sodium borohydride with a chiral organic acid such as camphoric acid or tartaric acid or an achiral acid such as succinic acid or phthalic acid (table 2, example 19).

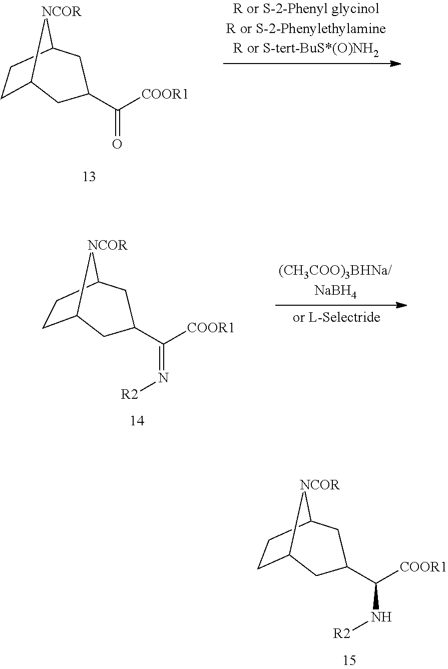

iii. conversion of compound of formula 15 to compound m first by treatment with methanolic-hydrogen chloride (MeOH—HCl), followed by treatment with di-tert-butyl dicarbonate ([(CH₃)₃COCO]₂O). Further, m can be converted to n and then to o by the known process.

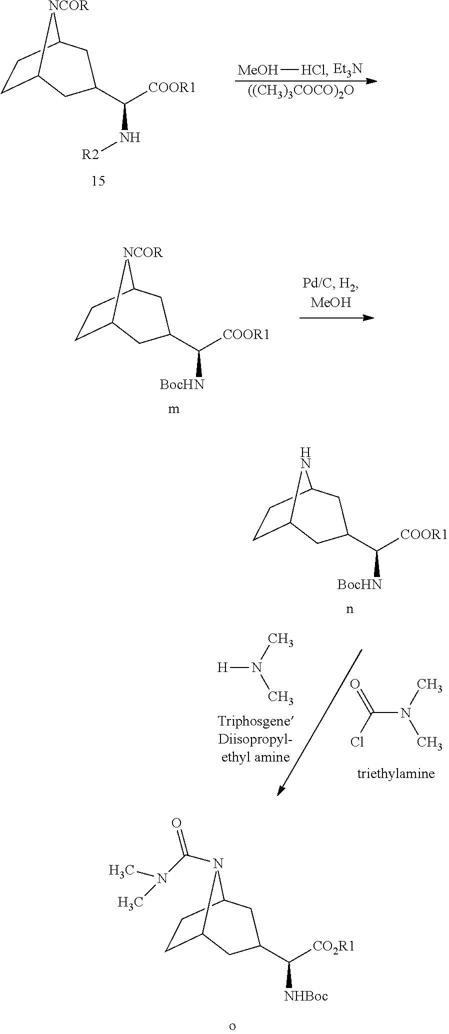

Such that at each step the intermediates were optionally isolated and purified with suitable process.

It is to be emphasized that the schemes 2, 3 and 4 starting compound is compound of to formula 1 and ends up in o only the difference is the “in between”

Compound of formula n or o can be further converted to DPP-IV inhibitors as described in WO 2009/037719 and PCT/IN2009/000478.

### DETAILED DESCRIPTION OF THE INVENTION

Present invention deals with novel, cost effective and eco-friendly process for synthesis of various important intermediates required for manufacture of DPP-IV inhibitors described in WO2009037719 and PCT/IN2009/000478 through novel compounds.

As shown in scheme 2, the key intermediate, benzyl 3-exo-formyl-8-azabicyclo[3.2.1]octane-8-carboxylate (compound 3) was obtained from benzyl 3-oxo-8-azabicyclo[3.2.1]octane-8-carboxylate (compound 1) using Corey-Chaykovsky reaction (Corey, E. J et al, J. Am. Chem. Soc, 1962, 84, 867-868) to give benzyl 8H-spiro[8-azabicyclo[3.2.1]octane-3,2′-oxirane]-8-carboxylate (compound 2), followed by epoxide opening using boron trifluoride-diethyl etherate (BF₃-etherate). In this reaction, a solution of benzyl 3-oxo-8-azabicyclo[3.2.1]octane-8-carboxylate (compound 1) in dimethyl formamide (DMF) was added to a solution of dimethylsulfonium ylide generated in situ by treating trimethylsulfonium iodide ((CH₃)₃S⁺I⁻) with sodium hydride (NaH) in dimethyl formamide (DMF). The reaction was carried out at room temperature and generally complete within an hour. The reaction mixture was then poured onto crushed ice and then extracted with ethyl acetate. Subsequently, the organic phase was washed with water followed by brine solution and then concentrated. The epoxide, benzyl 8H-spiro[8-azabicyclo[3.2.1]octane-3,2′-oxirane]-8-carboxylate (compound 2) was then opened to a mixture of exo and endo aldehydes, benzyl 3-formyl-8-azabicyclo[3.2.1]octane-8-carboxylate (compound 3) by using boron trifluoride-diethyl etherate (BF₃-etherate) in dichloromethane (DCM), employing Meinwald rearrangement (Meinwald J et al J. Am. Chem. Soc 1963, 85, 582-585) and the more thermodynamically stable exo aldehyde was obtained either by distillation or by column chromatography over silica gel or by treating with an appropriate base such as DBU.

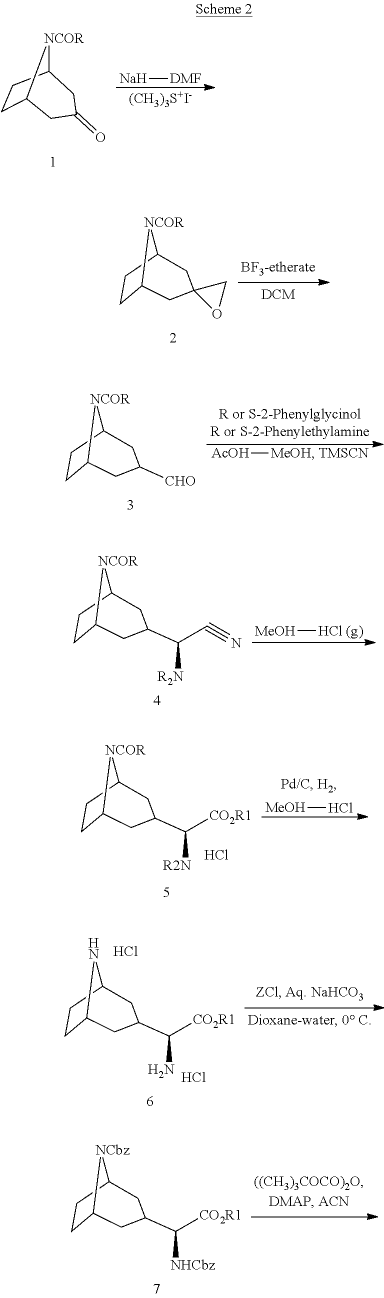

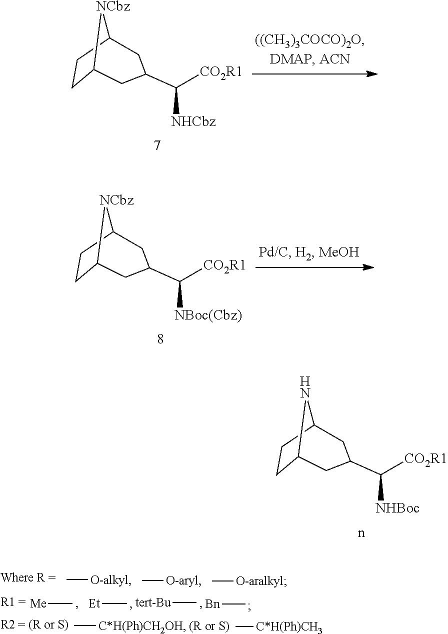

As shown in scheme 2, the exo-aldehyde, benzyl 3-exo-formyl-8-azabicyclo[3.2.1]octane-8-carboxylate (compound 3) in chloroform (or in methanol) was treated with chiral auxiliary namely, R-(−)-2-phenyl glycinol in the presence of acetic acid (AcOH) and the intermediate Schiff base formed in situ was treated with trimethyl silylcyanide (TMSCN) to give diasteromeric mixture of 1-(2-hydroxy-1-(1R)-phenylethyl amino)-1-(8-(benzyloxy carbonyl)-8-aza-bicyclo[3.2.1]-oct-3-yl)-exo-methane-1-(1S or 1R)-carbonitrile (4) (79:21; 1S,1′R; 1R,1′R). The diasteromeric mixture could be further separated either using column chromatography or by forming salts in general and hydrochloride salt in particular by treating the mixture with methanolic-hydrogen chloride (MeOH—HCl). The less soluble 1-(2-hydroxy-1-(1R)-phenylethyl amino)-1-(8-(benzyloxy carbonyl)-8-aza-bicyclo[3.2.1]-oct-3-yl)-exo-methane-1-(1S)-carbonitrile (4) crystallized as an off white solid.

Further improvement in diastereo selectivity was explored using different solvents and temperature as tabulated in Table 1. As shown in the table 1, the best selectivity was obtained by using chlorinated solvents in general and dichloro methane (DCM) in particular at room temperature (rt) (example 10). The enhanced diastereo selectivity by the chlorinated solvents over methanol and tetrahydrofuran (THF) is attributed to the stabilisation of the intermolecular hydrogen bonded transition state A by the former.

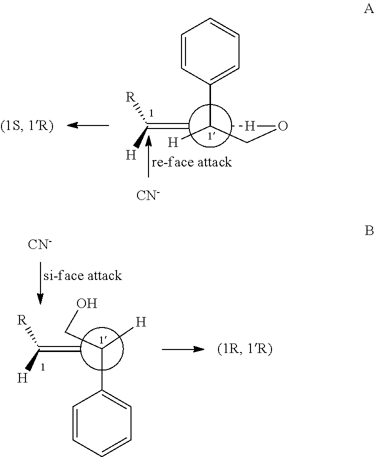

The major isomer in all the above experiments were assigned to be 1-(2-hydroxy-1-(1R)-phenylethyl amino)-1-(8-(benzyloxy carbonyl)-8-aza-bicyclo[3.2.1]-oct-3-yl)-exo-methane-1-(1S)-carbonitrile (compound 4).

An aspect of the present invention is that the aldehyde 3 could be converted to the intermediate 4 using other chiral amines such as (R or S) PhCH*(CH₃)NH₂ or any other chiral amine which bears an acid labile protection or an orthogonal protection, wherein, asterisk denotes point of attachment.

Transformation of the 1-(2-hydroxy-1-(1R)-phenylethyl amino)-1-(8-(benzyloxy carbonyl)-8-aza-bicyclo[3.2.1]-oct-3-yl)-exo-methane-1-(1S)-carbonitrile (compound 4) to the 1-(2-hydroxy-1-(1R)-phenylethyl amino)-1-(8-(benzyloxy carbonyl)-8-aza-bicyclo[3.2.1]-oct-3-yl)-exo-methane-1-(1S)-carboxylic acid methyl ester (compound 5) was carried out in methanolic-hydrogen chloride (3M, MeOH—HCl) in a single step. An aspect of the present invention is that, esters of the type 5 could also be prepared by converting the intermediate 4 to the corresponding acid, followed by esterification using alkyl or aralkyl alcohols. The above reaction mixture was then subjected to hydrogenolysis using an elevated pressure of hydrogen over palladium supported on charcoal (Pd/C) at room temperature (rt) or optionally at 60° C. to give the methyl (25)-2-amino-2-[8-azabicyclo[3.2.1]oct-3-yl]-exo-acetate hydrochloride (compound 6). Removal of the volatiles followed by treatment of the crude compound 6 in aqueous saturated sodium bicarbonate solution (satd. aq. NaHCO₃) with benzyl chloroformate (ZCl) at 0° C., afforded methyl (2S)-2-(benzyloxycarbonyl)-amino-2-[-8-[benzyloxycarbonyl]-8-azabicyclo[3.2.1]-oct-3-yl]-exo-acetate (compound 7). Compound 7 was subsequently transformed to methyl (2S)-2-((tert-butoxycarbonyl)-[benzyloxy carbonyl]-amino)-2-[8-(benzyloxy carbonyl)-8-azabicyclo-[3.2.1]oct-3-yl]-exo-acetate (compound 8) by treating with di-tert-butyldicarbonate ([(CH₃)₃COCO]₂O) in the presence of catalytic amount of 4-dimethylaminopyridine (DMAP). Hydrogenation of compound 8 over palladium supported on charcoal (Pd/C) at an elevated pressure of hydrogen afforded methyl (2S)-2-(tert-butoxycarbonyl)-amino-2-[-8-azabicyclo(3.2.1)oct-3-yl]-exo-acetate (compound n).

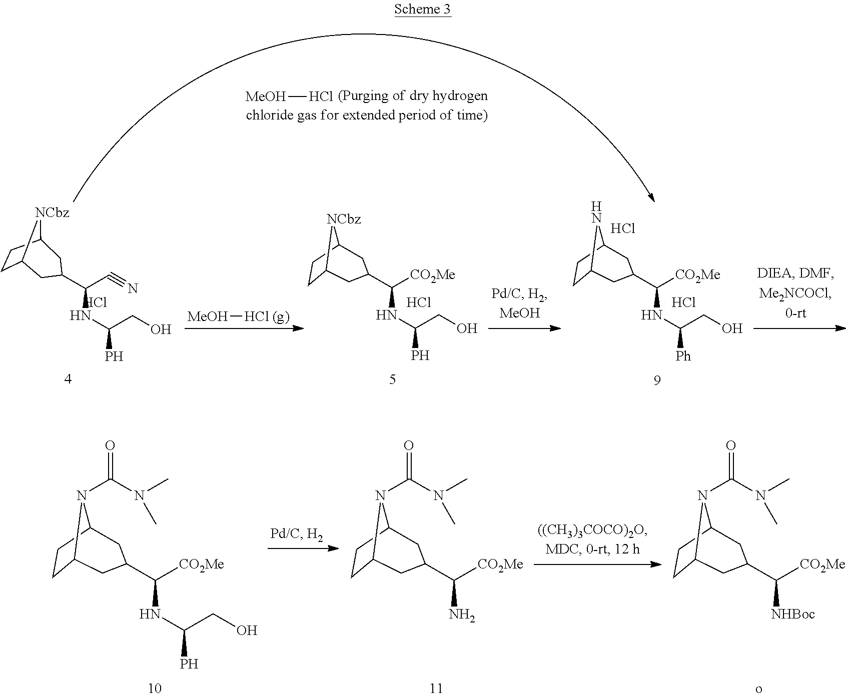

Scheme 3 illustrates the usefulness of the above strategy in preparing yet another advanced intermediate namely, methyl (2S)-2-(tert-butoxycarbonyl)-amino-2-[-8-[dimethylcarbamoyl]-8-azabicyclo[3.2.1]-oct-3-yl]-exo-acetate i.e. compound of formula o.

Thus, regio-selective hydrogenation of intermediate 5, over Pd supported on charcoal in methanol at ambient temperature and an elevated pressure of hydrogen afforded methyl (2S)-8-azabicyclo[3.2.1]-oct-3-yl[(2-hydroxy-1-(1R)-phenylethyl)-amino]-exo-acetate 9 in quantitative yield. Hydrogenolysis could also be effected either on the hydrochloride 5 or its acetate salt. It is also possible to carry out the hydrogenation of the free base generated by neutralizing 5 with either organic bases or with inorganic bases. Alternatively, compound 9 can be prepared from compound 4 by treating solution of compound 4 in methanol with dry hydrogen chloride for an extended period of time at room temperature. The intermediate 9 was subsequently converted to the 1-(2-hydroxy-1-(1R)-phenylethyl amino)-1-(8-(dimethyl carbamoyl)-8-aza-bicyclo[3.2.1]-oct-3-yl)-exo-methane-1-(1S)-carboxylic acid methyl ester 10 by treating with N,N dimethyl carbornyl chloride in the presence of an organic base such as diisopropyl ethylamine or triethylamine or with an inorganic base such as cesium carbonate in an organic solvents like dimethyl formamide, dimethyl acetamide, methylene dichloride, ethylene dichloride, cyclic or acyclic water miscible ethers or a combination of the above at 0° C. or at room temperature. Crude reaction mixture containing intermediate 10 and its diastereomers were separated either by column chromatography over silicagel column using a gradient of ethyl acetate in hexane or by crystallisation using solvent such as acetonitril or diethyl ether. Conversion of 10 to 11 was carried out by hydrogenolysis over Pd supported on charcoal in alcoholic solvents such as methanol contains acetic acid or methanolic hydrogen chloride at an elevated pressure of hydrogen at room temperature. Compound 11 was then converted to compound o by Boc protection of the resulting free amine using di-tert-butyl carbonate in dichloromethane (DCM) or ethylene dichloride (EDC) in the presence of an organic base such as triethylamine or disiopropylethylamine.

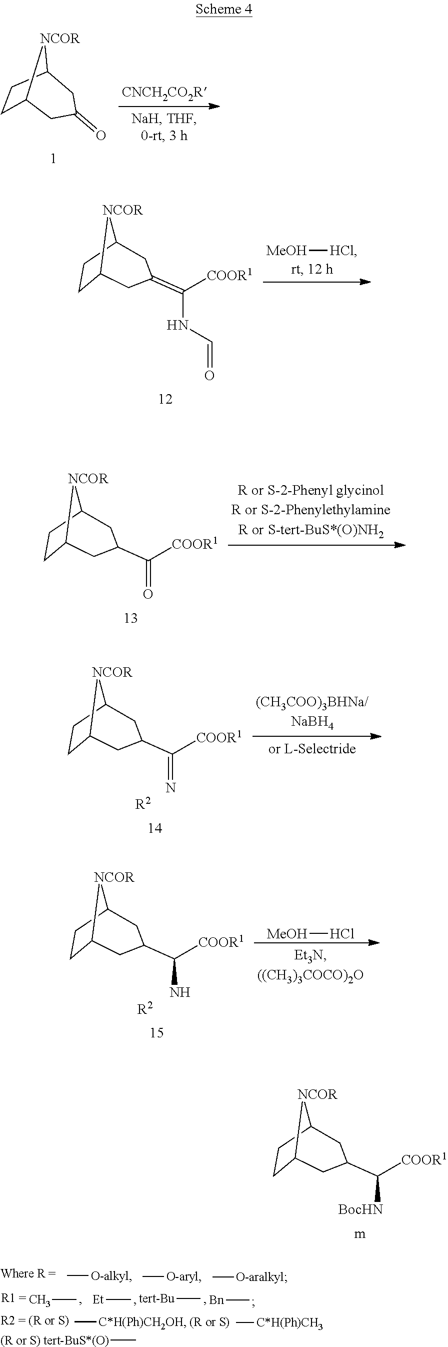

Alternatively, as shown in the scheme 4, intermediate benzyl 3-(1-formamido-2-ethoxy-2-oxoethylidene)-8-azabicyclo[3.2.1]-octane-8-carboxylate (compound 12) was prepared by the condensation of N-protected tropinone (wherein, bridge head N in 1 can be protected either as amides, ureas or as a urethanes), with an active methylene compounds such as alkyl or aryl or aralkyl isocyanoacetate in general and methyl or ethyl isocyanoacetate in particular.

Thus, the reaction of compound 1 with ethyl or methyl isocyanoacetate in tetrahydrofuran in the presence of a base such as sodium hydride (NaH) at 0° C. afforded the dehydro-amino acid derivative 12. The condensation could also be effected using organic bases such as DBU, BBN etc. Hydrolysis of the intermediate benzyl 3-(1-formamido-2-ethoxy-2-oxoethylidene)-8-azabicyclo[3.2.1]-octane-8-carboxylate (compound 12) to a mixture of ethyl or methyl esters, i.e. benzyl exo-3-[ethoxy/methoxy(oxo)acetyl]-8-azabicyclo[3.2.1]octane-8-carboxylate (compound 13) was carried out by treating 12 with methanolic-HCl. Hydrolysis of 12 to the keto ester 13 could also be possible using trifluoroacetic acid (TFA), hydrobromic acid (HBr) in an organic solvent or by using dilute aqueous hydrochloric acid. After evaporation of the solvents, the residue, benzyl exo-3-[ethoxy/methoxy(oxo)acetyl]-8-azabicyclo[3.2.1]octane-8-carboxylate (compound 13) was treated with either (S)-(−)-2-methyl-2-propane sulfinamide or with (R)-(−)-2-methyl-2-propane sulfinamide in the presence of titanium(IV) ethoxide (Ti(OEt)₄), in THF at 60° C. to give ketimine 14 which was then reduced to either 1-(S-tert-butylsulfinylamino)-1-(8-(benzyloxy carbonyl)-8-aza-bicyclo[3.2.1]-oct-3-yl)-exo-methane-1-(1S)-carboxylic acid ethyl ester or to 1-(R-tert-butylsulfinylamino)-1-(8-(benzyloxy carbonyl)-8-aza-bicyclo[3.2.1]-oct-3-yl)-exo-methane-1-(1S)-carboxylic acid ethyl ester (compound 15) using various reducing agents such as sodium acetoxy borohydride ((CH₃COO)₃BHNa), sodium borohydride (NaBH₄) or using any modified borohydrides derived from a combination of sodium borohydride with chiral acid such as camphoric acid or tartaric acid or with a achiral acid such as succinic or phthalic acid (table 2 example 19). The condensation of 14 to 15 could also be effected using dehydrating agents such as anhydrous copper sulfate or p-toluenesulfonic acid or by using pyridinium p-toluene sulfonate. Selective reduction of the ketimine 14 generated from 13 to either diastereomeric intermediate of 15, could also be possible by varying the use of chiral auxiliary or by changing the reducing agents such as L-selectride or hydrogenation over chiral catalyst such as Rhodium complexes or by using metal free catalytic reduction using trichlorosilanes in the presence of either achiral or chiral ligands. Cleavage of sulfinamide protection in 15 followed by conversion to the requisite intermediate m could be achieved by treating with dilute acids such as dilute hydrochloric acid or by treating with methanolic-hydrogen chloride (MeOH—HCl), followed by treating the free amine with di-tert-butyl dicarbonate ([(CH₃)₃COCO]₂O).

The following examples are provided to further illustrate the present invention and therefore should not be construed to limit the scope of the invention. All ¹H NMR spectra were determined in the solvents indicated and chemical shifts are reported in δ units downfield from the internal standard tetramethylsilane (TMS) and inter-proton coupling constants are reported in Hertz (Hz). The term ‘room temperature’ means a temperature anywhere between 20° C. to 40° C.

### Example 1

### Synthesis of benzyl 3-oxo-8-azabicyclo[3.2.1]octane-8-carboxylate (compound 1)

To a stirred solution of 8-methyl-8-azabicyclo[3.2.1]octan-3-one (200.0 g, 143 mmol) and K₂CO₃ (1.19 g) in toluene (4.0 L) was added benzyl chloroformate (1.96 L, 572 mmol, 50% in toluene) at room temperature and the resulting solution was stirred at reflux for 2 h. Reaction mixture was cooled to room temperature and then treated with ice-cold water (2.0 L). The organic phase was separated and the aqueous phase extracted with ethyl acetate (2.0 L×2). Organic phases were combined, dried over anhydrous sodium sulphate, filtered and evaporated to give a residue, which was purified by vacuum distillation. Yield: 215.0 g (55%). TLC, R_{f} (Hexane/Ethyl Acetate 30%)=0.3. IR cm⁻¹ (CHCl₃) 2959, 2887, 1702, 1414, 1367, 1338, 1320, 1284, 1156, 1004, 738. ¹HNMR (400 MHz, CDCl₃): δ 7.32 (m, 5H), 5.19 (s, 2H), 4.59 (br s, 2H), 2.72-2.56 (m, 2H), 2.34 (m, 2H), 2.1 (m, 2H), 1.68 (m, 2H). MS: 258 (M−1).

### Example 2

### Synthesis of benzyl 8H-spiro[8-azabicyclo[3.2.1]octane-3,2′-oxirane]-8-carboxylate (compound 2)

To a suspension of sodium hydride (34.5 g, 143 mmol, 50% suspension in hexane) in DMF (800 mL) was added, a solution of trimethyl sulfonium iodide (118.9 g, 0.58 mol) in DMF (800 mL), over a period of 1½ h. After 15 minutes at room temperature, a solution of benzyl 3-oxo-8-azabicyclo[3.2.1]octane-8-carboxylate (1, 100.0 g, 0.38 mol) in DMF (800 mL) was added. After stirring for 1 h, the reaction mixture was poured onto ice-cold water (6.4 L) and then extracted with ethyl acetate (2.0 L×3). Organic phases were combined, washed with water (1.0 L×3), followed by brine (1.0 L) and then dried over anhydrous sodium sulphate. It was filtered, the filtrate evaporated and the residue was directly used for the next step. Yield: 105 g. TLC, R_{f} (Hexane/Ethyl Acetate 30%)=0.35. IR cm⁻¹ (CHCl₃) 2950, 1698, 1414, 1322, 1204, 1097, 1008, 760. ¹HNMR (400 MHz, CDCl₃): δ 7.32 (m, 5H), 5.15 (s, 2H), 4.41 (m, 2H), 2.43 (s, 2H), 2.38 (m, 2H), 2.13-1.99 (m, 4H), 1.22 (m, 2H). MS: 274 (M+1)⁺.

### Example 3

### Synthesis of benzyl 3-exo-formyl-8-azabicyclo[3.2.1]octane-8-carboxylate (compound 3)

To a stirred solution of benzyl 8H-spiro[8-azabicyclo[3.2.1]octane-3,2′-oxirane]-8-carboxylate (2, 100.0 g, 0.36 mol) in dichloromethane (1.2 L) at room temperature, was added BF₃-etherate (26.0 g, 0.18 mol), drop wise, over a period of ½ h. Stirring was continued for 2 h and the reaction mixture was treated with ice-cold water (500 mL). Organic phase was separated and washed with saturated sodium bicarbonate solution (500 mL), followed by brine (500 mL). It was dried over anhydrous sodium sulphate, filtered and concentrated under vacuo to give a residue, which was purified over silica gel column using a gradient of ethyl acetate in hexane. Yield: 70.6 g (71%). TLC, R_{f} (Hexane/Ethyl Acetate 30%)=0.3. IR cm⁻¹ (CHCl₃) 2954, 1698, 1422, 1328, 1213, 1102, 1078, 751. ¹HNMR (400 MHz, CDCl₃): δ 9.54 (s, 1H), 7.33 (m, 5H), 5.14 (s, 2H), 4.39 (m, 2H), 2.82 (m, 1H), 2.06-1.65 (m, 8H). MS: 274 (M+1)⁺.

### Example 4

### Synthesis of 1-(2-hydroxy-1-(1R)-phenylethyl amino)-1-(8-(benzyloxy carbonyl)-8-aza-bicyclo[3.2.1]-oct-3-yl)-exo-methane-1-(1S)-carbonitrile hydrochloride salt (compound 4)

To a stirred solution of benzyl 3-exo-formyl-8-azabicyclo[3.2.1]octane-8-carboxylate (3, 98.0 g, 0.35 mol) in methanol (686 mL) under nitrogen, was added R-(−)-2-phenyl glycinol (49.2 g, 0.35 mol) followed by glacial acetic acid (22 mL), drop wise, over a period of 5 min. After ½ h at room temperature, trimethylsilyl cyanide (39.1 g, 0.39 mol) was added dropwise. Stirring was continued for an additional 2 h and the reaction quenched by treating with DM water (686.0 mL). Volatiles were removed under vacuum and the residue extracted with ethyl acetate (1.5 L×2). Organic phases were combined and the combined extract washed with DM water (1.0 L) followed by brine (1.0 L) and then dried over anhydrous sodium sulphate. It was filtered, concentrated in vacuo and the residue was dissolved in methanolic hydrogen chloride (4M, 300 mL). Into this was added diethyl ether (500 mL) and the mixture kept at room temperature overnight. The white solid separated was filtered, washed with diethyl ether and dried. Yield=63.0 g (42%). TLC, R_{f} (Hexane/Ethyl Acetate 40%)=0.4. IR cm⁻¹ (CHCl₃) 3248, 2951, 1686, 1454, 1425, 1326, 1212, 1103, 757. ¹HNMR (400 MHz, CDCl₃): δ 7.33 (m, 10H), 5.13 (s, 2H), 4.36 (m, 2H), 4.07 (dd, J=3.6, 9.2 Hz, 1H), 3.77 (dd, J=3.6, 10.8, 1H), 3.53 (t, J=10 Hz, 1H), 3.02 (br s, 1H), 2.17 (br m, 1H), 2.05-1.80 (m, 4H), 1.74-1.41 (m, 4H). MS: 420 (M+1)⁺.

### Example 5

### Synthesis of 1-(2-hydroxy-1-(1R)-phenylethyl amino)-1-(8-(benzyloxy carbonyl)-8-aza-bicyclo[3.2.1]-oct-3-yl)-exo-methane-1-(1S)-carboxylic acid methyl ester. hydrochloride salt (compound 5)

A stirred suspension of 1-(2-hydroxy-1-(1R)-phenylethyl amino)-1-(8-(benzyloxy carbonyl)-8-aza-bicyclo[3.2.1]-oct-3-yl)-exo-methane-1-(1S)-carbonitrile hydrochloride salt (4, 45.0 g, 0.107 mol) in methanol (540 mL) was heated at 70° C. till it become a clear solution. It was brought to room temperature and dry hydrogen chloride gas was purged until the molarity of the solution become 3. It was kept at room temperature for 24 h and then concentrated to give a semi solid which was finally co-evaporated with toluene to give a free flowing solid which was contaminated with benzyloxy carbonyl deprotected 5. An analytical sample was prepared by purifying the above reaction mixture by column chromatography over silica gel using a gradient of ethyl acetate in hexane. IR cm⁻¹ (CHCl₃) 3320, 2923, 1698, 1533, 1420, 1327, 1101, 784. ¹HNMR (400 MHz, CDCl₃): δ 7.30 (m, 10H), 5.12 (s, 2H), 4.30 (m, 3H), 3.69 (s, 3H), 3.60 (m, 2H), 2.83 (d, J=7.2 Hz, 1H), 1.5-2.1 (m, 8H). MS: 453 (M+1)⁺.

### Example 6

### Synthesis of methyl (2S)-2-amino-2-[8-azabicyclo[3.2.1]oct-3-yl]-exo-acetate, hydrochloride salt (compound 6)

A solution of 1-(2-hydroxy-1-(1R)-phenylethyl amino)-1-(8-(benzyloxy carbonyl)-8-aza-bicyclo[3.2.1]-oct-3-yl)-exo-methane-1-(1S)-carboxylic acid methyl ester hydrochloride salt (5, 45.0 g, 90 mmol) in methanolic-hydrogen chloride (450 mL, 1M), was hydrogenated over palladium supported on charcoal (20%, 22.5 g) using a positive pressure of hydrogen (20 Kg) at 60° C. for 12 h. It was then filtered through a celite pad and washed with methanol (250 mL). The filtrate was concentrated in vacuo to give a pale yellow semi solid, which was used for the next step without further purification. ¹HNMR (400 MHz, DMSO-d6): δ 9.34 (s, 1H), 8.81 (s, 2H), 3.96 (br s, 2H), 3.76 (s, 3H), 1.97-1.83 (m, 3H), 1.20-1.10 (m, 6H). MS: 199 (M+1)⁺.

### Example 7

### Synthesis of methyl (2S)-2-(benzyloxycarbonyl)-amino-2-[-8-benzyloxy carbonyl]-8-azabicyclo[3.2.1]oct-3-yl]exo-acetate (compound 7)

Methyl (2S)-2-amino-2-[8-azabicyclo[3.2.1]oct-3-yl]-exo-acetate, hydrochloride salt (6, 23.0 g, 80 mmol) was dissolved in a mixture of DM water and 1,4 dioxane (230 mL, 1:1). The resulting solution was then cooled to 0° C. and was treated with aqueous saturated sodium bicarbonate till the pH of the solution become 7.5. Into this was added benzyl chloroformate (42.19 g, 0.24 mol, 50% in toluene), drop wise, such that pH remains around 7.5. It was stirred for 3 h at 0° C. and then extracted with ethyl acetate (500 mL×3). Organic phases were combined and the combined phase was washed with DM water (500 mL) followed by brine (500 mL) and then dried over anhydrous sodium sulphate. It was filtered, concentrated in vacuo and the residue was purified over silica gel column using a gradient of ethyl acetate in hexanes to give colourless oil. Yield=21.2 g (55%). TLC, R_{f} (Hexane/Ethyl Acetate 40%)=0.3. ¹HNMR (400 MHz, CDCl₃): δ 7.34 (m, 10H), 5.29 (m, 11-1), 5.12 (s, 2H), 5.03 (s, 2H), 4.26 (m, 3H), 3.71 (s, 3H), 2.33 (m, 1H), 1.96 (m, 2H), 1.64-1.25 (m, 6H). MS: 467 (M+1)⁺.

### Example 8

### Synthesis of methyl (2S)-2-((tert-butoxycarbonyl)-[benzyloxy carbonyl]-amino)-2-[8-(benzyloxy carbonyl)-8-azabicyclo[3.2.1]oct-3-yl]-exo-acetate (compound 8)

To a stirred solution of methyl (2S)-2-(benzyloxycarbonyl)-amino-2-[-8-[benzyloxy carbonyl]-8-azabicyclo[3.2.1]oct-3-yl]-exo-acetate (7, 21.0 g, 40 mmol) and DMAP (1.09 g, 9 mmol) in acetonitrile (210 mL) was added di-tert-butyl dicarbonate (23.57 g, 108 mmol) at room temperature. It was stirred for 12 h and then treated with DM water (420 mL) and extracted with ethyl acetate (0.5 L×2). Organic phases were combined; combined phase was washed with DM water (250 mL×2), followed by brine (0.25 L×2) and then dried over anhydrous sodium sulfate. It was filtered and concentrated in vacuo. The residue thus obtained was purified over silica gel column using a gradient of ethyl acetate in hexanes to give pale yellow colored oil. Yield=24.3 g (76%). TLC, R_{f} (Hexane/Ethyl Acetate 40%)=0.4. IR cm⁻¹ (CHCl₃) 2976, 1750, 1701, 1395, 1233, 1151, 1105, 753. ¹HNMR (400 MHz, CDCl₃): δ 7.34 (m, 10H), 5.26 (m, 2H), 5.13 (s, 2H), 4.61 (d, J=9.2 Hz, 1H), 4.30 (m, 2H), 3.64 (s, 3H), 2.75 (m, 1H), 2.17 (m, 1H), 1.91 (m, 2H), 1.71 (m, 1H), 1.58-1.33 (m, 5H). MS: 467 (M−99)⁺, 589 (M+Na)⁺.

### Example 9

### Synthesis of methyl (2S)-2-(tert-butoxycarbonyl)-amino-2-[-8-azabicyclo[3.2.1]oct-3-yl]-exo-acetate (compound n)

A solution of methyl (2S)-2-((tert-butoxycarbonyl)-[benzyloxy carbonyl]-amino)-2-[8-(benzyloxy carbonyl)-8-azabicyclo[3.2.1]oct-3-yl]exo-acetate (8, 3.5 g, 6.1 mmol) was hydrogenated over palladium supported on charcoal (10%, 0.35 g) using a positive pressure of hydrogen (5 Kg) at room temperature for 2 h. It was the filtered through a celite pad and the filtrate was concentrated to dryness. Yield: 1.50 g (89%). IR cm⁻¹ (CHCl₃) 3445, 1644, 1519, 1367, 116, 754. ¹HNMR (400 MHz, CDCl₃+D₂O): δ 4.19 (d, J=9.6 Hz, 1H), 3.72 (s, 3H), 3.56 (brs, 2H), 2.10-2.22 (m, 1H), 1.70-1.85 (m, 2H), 1.52-1.68 (m, 2H), 1.30-1.50 (m, 13H). MS: 299 (M+1)⁺. [α]^{D}₂₀: −3.74 (c=1, MeOH)

### Example 10

### Synthesis of 1-(2-hydroxy-1-(1R)-phenylethyl amino)-1-(8-(benzyloxy carbonyl)-8-aza-bicyclo[3.2.1]-oct-3-yl)-exo-methane-1-(1S)-carbonitrile (compound 4)

To a stirred solution of benzyl 3-exo-formyl-8-azabicyclo[3.2.1]octane-8-carboxylate (3, 50 mg, 0.18 mmol) in dichloromethane (3.5 mL) under nitrogen at ambient temperature, was added R-(−)-2-phenyl glycinol (30 mg, 0.22 mmol) followed by glacial acetic acid (0.1 mL). After ½ h at room temperature, trimethylsilyl cyanide (20 mg, 0.22 mmol) was added and stirring continued for 2 h. The reaction was quenched by treating with DM water (3.0 mL) and evaporated under vacuum. The residue was extracted with ethyl acetate (3.0 mL×2), washed with DM water (3.0 mL) followed by brine (3.0 mL) and then dried over anhydrous sodium sulfate. It was filtered, concentrated and the residue analyzed by reverse phase HPLC.

TABLE 1

| | | | Diastereo selectivity |
|---|---|---|---|
| Sr. No. | Solvent | Temp ° C. | (1R,1′R:1S,1′R) |
| 1 | CH₂Cl₂ | 25 | 19:81 |
| | | 0 | 29:71 |
| | | −20 | 26:77 |
| | | −40 | 38:62 |
| 2 | MeOH | 25 | 28:72 |
| 3 | Hexane | 25 | 41:58 |
| 4 | CHCl₃ | 25 | 22:78 |
| 5 | Methyl THF | 25 | 34:66 |
| 6 | THF | 25 | 39:60 |
| 7 | DME | 25 | 35:65 |
| 8 | CH₂ClCH₂Cl | 25 | 21:79 |

Method and analysis: Analytical reversed-phase HPLC was performed on a Supelco C-18 column (5×4.6 mm, 2.7μ), eluted with a gradient of acetonitrile (MeCN) in water (containing 20 mM KH₂PO₄, pH=3.0), flow rate 0.7 mL/min, detection at 210 nm.

### Example 11

### Synthesis of 1-(2-hydroxy-1-(1R)-phenylethyl amino)-1-(8-(dimethyl carbamoyl)-8-aza-bicyclo[3.2.1]-oct-3-yl)-exo-methane-1-(1S)-carboxylic acid methyl ester (compound 10)

A solution of crude 1-(2-hydroxy-1-(1R)-phenylethyl amino)-1-(8-(benzyloxy carbonyl)-8-aza-bicyclo[3.2.1]-oct-3-yl)-exo-methane-1-(1S)-carboxylic acid methyl ester. hydrochloride salt (5, 54.0 g, 119 mmol) in methanol (540 mL) was hydrogenated over Pd(0)/C (10%, 11 g) at an elevated pressure of hydrogen at room temperature for 3 h during which tlc showed the disappearances of 5. It was filtered through celite pad and the filtrate concentrated to dryness to give a solid (intermediate 9) which was dissolved in DMF (300 mL). It was cooled to 0° C. and then treated with diisopropyl ethylamine (47.19 mL, 364 mmol). After stirring for 15 min at 0° C., reaction mixture treated with a solution of N,N dimethyl carbonyl chloride (13.48 g, 124 mmol) in DMF (40 mL). It was allowed to reach room temperature and stirring continued for 16 h during which tlc showed the completion of reaction. Reaction was quenched by the addition of dry methanol (15 mL) followed by water (200 mL). It was extracted with tert-butyl methyl ether (200 mL×5), organic phases combined and the combined phase washed with brine (200 mL). Organic phase separated, dried over anhydrous sodium sulfate, filtered and the filtrate concentrated to dryness. The residue thus obtained was treated with diethyl ether to give a white solid. Yield: 15.0 g to (32%). MP: 123.7° C., HPLC purity 99% (Analytical reversed-phase HPLC was performed on a ascentis C-18 column (5×4.6 mm, 2.7μ), eluted with a gradient of acetonitrile (MeCN) in water (containing 20 mM KH₂PO₄, pH=3.0), flow rate 0.7 mL/min, detection at 210 nm). [α]^{D}₂₀: −16.60° (c=1, CHCl₃). IR (KBr) 3354, 3296, 2990, 2944, 2861, 1733, 1640, 1489, 1450, 1385, 1231, 1199, 1167, 1060, 1020, 917, 807, 706. ¹HNMR (400 MHz, dmso-d6): δ 7.24-7.33 (m, 5H), 5.03 (m, 1H), 3.97 (brs, 1H), 3.88 (brs, 2H), 3.68 (s, 3H), 3.46 (m, 1H), 3.25 (m, 1H), 2.74 (s, 6H), 2.55 (brs, 2H), 1.82-1.89 (m, 2H), 1.65-1.80 (m, 2H), 1.35-1.60 (m, 3H), 1.10-1.30 (m, 2H). MS: 390 (M+1)⁺.

### Example 12

### Synthesis of methyl (2S)-8-azabicyclo[3.2.1]oct-3-yl[(2-hydroxy-1-(1R)-phenylethyl)amino]-exo-acetate hydrochloride salt (compound 9)

A stirred suspension of 1-(2-hydroxy-1-(1R)-phenylethyl amino)-1-(8-(benzyloxy carbonyl)-8-aza-bicyclo[3.2.1]-oct-3-yl)-exo-methane-1-(1S)-carbonitrile hydrochloride salt (4, 109 g, 239 mmol) in methanol (1.6 L) was heated at 70° C. till it became a clear solution. It was brought to room temperature and dry hydrogen chloride gas was purged for period of 8 h during which the hydrolysis of the cyano group gets completed. It was kept at room temperature for an additional 24 h which resulted in the complete deprotection of benzyloxy carbonyl group. Into this nitrogen gas was bubbled and the mixture evaporated to dryness to give the hydrochloride salt 9. MS: 319 (M+1)⁺. The so obtained residue of compound 9 was further converted to compound 10 by using the procedure provided in example 11 for conversion of compound 9 to 10.

### Example 13

### Synthesis of methyl (2S)-2-(tert-butoxycarbonyl)-amino-2-[-8-[dimethylcarbamoyl]-8-azabicyclo[3.2.1]-oct-3-yl]-exo-acetate (compound o).

A solution of 1-(2-hydroxy-1-(1R)-phenylethyl amino)-1-(8-(dimethyl carbamoyl)-8-aza-bicyclo[3.2.1]-oct-3-yl)-exo-methane-1-(1S)-carboxylic acid methyl ester (compound 10, 4.0 g, 10.2 mmol) and acetic acid (8 mL) in methanol (32 mL) was hydrogenated over Pd/C (10%, 1.0 g) using a positive pressure of hydrogen (20 Kg). After 2 h, tlc showed the disappearance of 10. It was filtered through celite pad and the filtrate concentrated to dryness to obtain (S)-Amino-(8-dimethylcarbamoyl-8-aza-bicyclo[3.2.1]oct-3-yl)-acetic acid methyl ester 11 [m/z=270 (M+1)]. The residue (compound 11, 5.0 g) thus obtained was dissolved in MDC (27 mL), cooled to 0° C. and treated with triethylamine (4.2 mL, 3.0 mmol). Into this was added di-tert-butyl dicarbonate (2.61 g. 1.2 mmol) and the mixture stirred overnight. Solvents were evaporated and the residue treated with water (10 mL). It was extracted with dichloromethane (3×20 mL), organic phases combined and the combined phase washed with brine (10 mL) and dried over anhydrous sodium sulphate. It was filtered and the filtrate concentrated. The residue thus obtained was purified over silica gel column using a gradient of EtOAc in hexane to afford o as a gummy solid. Yield: 3.0 g (79%). IR cm⁻¹ (CHCl₃) 3304, 2949, 1745, 1713, 1630, 1495, 1445, 1365, 1295, 1259, 1165, 1062, 1023, 900, 863, 754. ¹HNMR (400 MHz, CDCl₃): δ 5.024 (m, 1H), 4.22 (m, 1H), 4.08 (m, 2H), 3.74 (s, 3H), 2.86 (s, 6H), 2.21 (m, 1H), 1.89 (m, 2H), 1.72 (m, 1H), 1.60 (m, 4H), 1.43 (s, 9H). MS: 370 (M+1)⁺. [α]^{D}₂₀: +9.52° (c=1, CHCl₃)

### Example 14

### Synthesis of benzyl 3-(1-formamido-2-ethoxy-2-oxoethylidene)-8-azabicyclo[3.2.1]-octane-8-carboxylate (compound 12)

To a stirred solution of sodium hydride (47.0 g, 1.59 mol) in tetrahydrofuran (5.0 L) was added a solution of ethyl isocyanoacetate (192 g, 1.41 mol) in tetrahydrofuran (1.2 L), drop wise, over a period of 30 min at 0-10° C. After 45 minutes at this temperature, a solution of benzyl 3-oxo-8-azabicyclo[3.2.1]-octane-8-carboxylate (1, 335 g, 1.08 mol), in tetrahydrofuran (1.25 L) was added dropwise over a period of 30 min. It was stirred for 1 h at 0-10° C. and the reaction quenched by treating with crushed ice. Reaction mass extracted with ethyl acetate (2×5 L), organic phases combined and the combined organic phase washed with brine (5 L). It was dried over anhydrous sodium sulfate, filtered and concentrated under vacuum. The residue was re-crystallized from ethyl acetate/Hexane to give a colorless solid. Yield: 345 g (76%). MP: 124.5° C. IR cm⁻¹ (CHCl₃) 3288, 989, 1705, 1506, 1422, 1311, 1203, 1092, 1036, 754. ¹HNMR (400 MHz, CDCl₃) δ: 8.17 (s, 1H), 7.37 (br s, 5H), 5.19 (s, 2H), 4.40 (br s, 2H), 4.20 (q, 2H, J=7.14 Hz), 2.31 (m, 3H), 1.70 (m, 3H), 1.50 (m. 2H), 1.29 (t, 3H, J=7.14 Hz). MS: 373 (M+1)⁺, 390 (M+H₂O)⁺, 395 (M+Na)⁺

### Example 15

### Synthesis of benzyl exo-3-[ethoxy/methoxy(oxo)acetyl]-8-azabicyclo-[3.2.1]octane-8-carboxylate (compound 13)

To a solution of benzyl 3-(1-formamido-2-ethoxy-2-oxoethylidene)-8-azabicyclo[3.2.1]-octane-8-carboxylate (12, 100 g, 0.27 mol) in methanol (1 L) at 0° C., was added methanolic-hydrogen chloride (200 mL, 4M) and the solution stirred for 12 h. It was concentrated and the residue dissolved in ethyl acetate (1.0 L) and dried over anhydrous sodium sulfate. It was filtered and concentrated. The residue re-dissolved in dichloromethane (2.0 L) and treated with charcoal (30 g). It was filtered and concentrated. The residue which contains a mixture of ethyl and methyl ester was used as such for the next step without further purification. Yield: 82.0 g (92%). IR cm⁻¹ (CHCl₃) 3423, 1644, 1417, 1096, 770. ¹HNMR (400 MHz, CDCl₃) δ: 7.34 (s, 5H), 5.18 (s, 2H), 4.40 (brs, 2H), 4.20-4.50 (m, 3H), 3.85 (s, 2H), 3.55 (m, 1H), 1.62-2.00 (m, 8H), 1.28 (m, 2H). MS: 330 (M−1).

### Example 16

### Synthesis of Benzyl 3-[(1E/Z)-N-((S)-tert-butylsulfinyl)-2-ethoxy-2-oxoethanimidoyl]-8-azabicyclo[3.2.1]octane-8-carboxylate (compound 14).

To a stirred solution of mixture of benzyl exo-3-[ethoxy or methoxy(oxo)acetyl]-8-azabicyclo[3.2.1]octane-8-carboxylate (13, 20.0 g, 0.06 mol) in tetrahydrofuran (600 mL) was added S-(−)-tert-butyl sulfinamide (9.54 g, 0.078 mol) followed by Ti(OEt)₄ (27.63 g, 0.12 mol) and the resulting solution was refluxed for 12 h during which tlc showed the disappearance of 13. It was diluted with a mixture of ethyl acetate:water (2:1, 1.8 L), filtered through celite bed. Layers were separated and dried over anhydrous sodium sulfate. It was filtered and the filtrate concentrated. The residue re-dissolved in dichloromethane (500 mL) and treated with charcoal (20 g) at 40° C. It was filtered through celite pad and the filtrate treated with neutral alumina (40 g) for a period of 45 min. Solids was filtered, washed with dichloromethane and the filtrate evaporated. Yield: 19.2 g (70%). MP: 99.4° C. IR cm⁻¹ (KBr) 2976, 1738, 1706, 1624, 1452, 1404, 1258, 1079, 975, 870, 743. ¹HNMR (400 MHz, CDCl₃) δ: 7.31 (5H, brs), 5.17 (s, 2H), 4.20-4.50 (m, 4H), 3.08 (m, 1H), 2.02 (m, 3H), 1.54 (m, 6H), 1.35 (t, J=6.3 Hz, 3H), 1.22 (s, 9H). MS: 449 (M+1)⁺.

### Example 17

### Synthesis of 1-(S-tert-butylsulfinylamino)-1-(8-(benzyloxy carbonyl)-8-aza-bicyclo[3.2.1]-oct-3-yl)-exo-methane-1-(1S)-carboxylic acid ethyl ester (compound 15)

To a solution of benzyl 3-[(1E/Z)-N-((S)-tert-butyl sulfinyl)-2-ethoxy-2-oxoethanimidoyl]-8-azabicyclo[3.2.1]octane-8-carboxylate (14, 5.0 g, 11.5 mmol) in tetrahydrofuran (50 mL) at room temperature was added triacetoxy borohydride (14.61 g, 67 mmol) and mixture stirred for 24 h. Reaction quenched by treating with methanol (25 mL) followed by water (50 mL) and extracted with ethyl acetate (3×50 mL). Organic phases combined, washed with water followed by brine and then dried over anhydrous sodium sulfate. It was filtered and the filtrate concentrated to dryness. The residue thus obtained was used directly for the next step. Yield: 5.30 g. IR cm⁻¹ (KBr) 3462, 3235, 2957, 1739, 1682, 1447, 1417, 1331, 1263, 1102, 1070, 1027, 893, 764, 735. ¹HNMR (400 MHz, CDCl₃) δ: 7.33 (brs, 5H), 5.12 (s, 2H), 4.35 (brs, 2H), 4.13 (m, 2H), 3.66 (2H, m), 2.23 (m, 1H), 1.96 (brs, 2H), 1.63 (brs, 6H), 1.25 (m, 3H), 1.18 (s, 9H). MS: 451 (M+1)⁺.

### Example 18

### Synthesis of ethyl (2S)-2-((tert-butoxycarbonyl)-amino)-2-[8-(benzyloxy carbonyl)-8-azabicyclo[3.2.1]oct-3-yl]-exo-acetate (compound m)

A solution of 1-(S-tert-butylsulfinylamino)-1-(8-(benzyloxy carbonyl)-8-aza-bicyclo[3.2.1]-oct-3-yl)-exo-methane-1-(1S)-carboxylic acid ethyl ester (compound 15, 5.3 g, crude) in methanol (50 mL) was added methanolic-hydrogen chloride (10 mL, 3M) and the resulting solution stirred at room temperature for 3 h during which tlc showed the disappearance of the starting material. It was concentrated and dried. It was dissolved in tetrahydrofuran (74 mL) and treated with triethylamine till the pH of the solution was neutral. Into this was added di-tert-butyl dicarbonate (2.95 g) and the reaction mixture stirred for 12 h. Reaction was quenched by adding water (74 mL) and extracted with dichloromethane (3×75 mL). Organic phases combined, washed with water and dried over anhydrous sodium sulfate. It was filtered and the filtrate evaporated to dryness. The residue was purified over silica gel column using a gradient of ethyl acetate in petroleum ether to give m as gummy solid. Yield: 4.0 g (82%). IR cm⁻¹ (KBr) 3337, 2972, 2927, 1702, 1526, 1457, 1333, 1171, 1098, 1020, 864, 755. 754. ¹HNMR (400 MHz, CDCl₃) δ: 7.35 (brs, 5H), 5.13 (s, 2H), 5.00 (m, 1H), 4.35 (m, 2H), 4.18 (m, 2H), 2.32 (m, 1H), 1.96 (m, 2H), 1.52-1.73 (m, 6H), 1.45 (s, 9H), 1.26 (t, J=7.2 Hz, 3H). MS: 348 (M-Boc)⁺, 447 (M+1)⁺.

### Example 19

A suspension of sodium borohydride (212.0 mg, 5.6 mmol) and (1R,3S)-camphoric acid (8.2 mmol) or D-tartaric acid (8.2 mmol) in THF (12 mL) was refluxed for 2 h under nitrogen atmosphere. The above solution was cooled and added a solution of ketimine 14 (500 mg, 11 mmol) in THF (4 mL) and stirred for a period of 12 h at room temperature. Reaction was quenched by treating with saturated sodium bicarbonate solution (30 mL) and the content was extracted into dichloromethane (10 mL×2). Organic phases combined, combined phase washed with water (10 mL×2), followed by brine (10 mL), dried over anhydrous sodium sulfate and evaporated to give pale yellow oil (˜510 mg).

TABLE 2

| | NaBH₄: | NaBH₄: | |
|---|---|---|---|
| Temperature | (1R,3S)-Camphoric | D-Tartaric acid | Diastereomer ratio |
| (° C.) | acid (Mole ratio) | (Mole ratio) | (SR:RR) |
| rt | 1:1 | — | 84:16 |
| −30 | 1:1 | — | 84:16 |
| 0 | 1:1 | — | 87:13 |
| rt | 1:1 | — | 87:13 |
| −30 | 1:1.5 | — | 88:12 |
| 0 | 1:1.5 | — | 88:12 |
| rt | 1:1.5 | — | 88:12 |
| 40 | 1:1.5 | — | 88:12 |
| rt | — | 1:1 | 88:12 |
| rt | — | 1:5 | 91:09 |

Method and analysis: Analytical reversed-phase HPLC was performed on a cosmosil cholester (250×4.6 mm, 5μ), eluted with a gradient of acetonitrile (MeCN) in water (flow rate 1 mL/min, detection at 210 nm).

We claim:

1. A process for the synthesis of compound of formula n 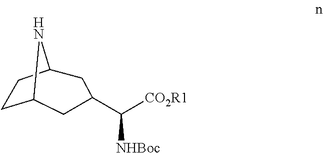
wherein, R1 is selected from C1 to C4 alkyl or benzyl; comprising:
(i) conversion of compound of formula 1 to compound of formula 3 by first converting compound of formula 1 to an oxiranyl compound 2 by treatment with sodium hydride and trimethylsulfonium iodide in N,N-dimethylformamide at temperature between 20 to 40° C. and further to compound of formula 3 by treatment with BF₃-etherate in dichloromethane at room temperature; 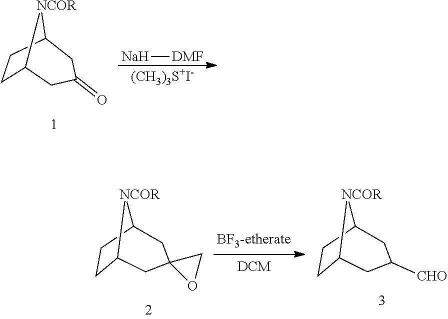
Wherein, R is selected from substituted or unsubstituted phenyl, C1 to C4 alkyloxy, substituted or unsubstituted benzyloxy;
(ii) conversion of compound of formula 3 to compound of formula 4 following the Strecker synthesis using chiral amine such as R-(−)-2-Phenyl glycinol or R-(−)-2-phenylethylamine or their optical isomers as a chiral auxiliary and TMSCN as a cyanide source in an organic solvent selected from dichloromethane, methanol, chloroform, 1,2-ethylenedichloride, hexane, tetrahydrofuran, methyltetrahydrofuran, DME or mixtures thereof in the presence of acetic acid, or Lewis acid at a temperature between −40 and 25° C.; 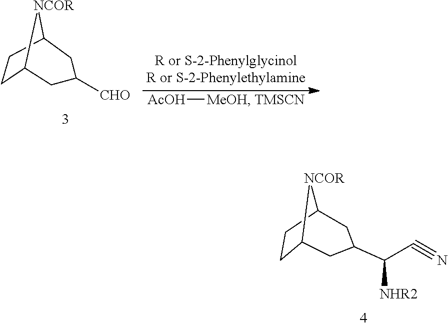
Wherein, R is selected from substituted or unsubstituted phenyl, C1 to C4 alkyloxy, substituted or unsubstituted benzyloxy; and R2 is selected from (R/S)—C*H(Ph)CH₂OH, (R/S)—C*H(Ph)CH₃, wherein, asterisk denotes point of attachment;
(iii) hydrolysis of the nitrile group in compound of formula 4 using alcoholic-HCl to obtain compound of formula 5 at room temperature; 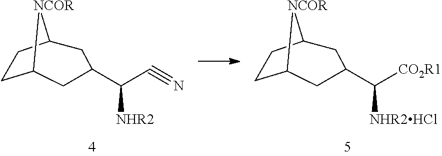
Wherein, R is selected from substituted or unsubstituted phenyl, C1 to C4 alkyloxy, substituted or unsubstituted benzyloxy; R1 is selected from C1 to C4 alkyl or benzyl; and R2 is selected from (R/S)—C*H(Ph)CH₂OH, (R/S)—C*H(Ph)CH₃ wherein, asterisk denotes point of attachment;
(iv) conversion of compound of formula 5 to compound of formula 6 by the de-protection of amino group in presence of palladium supported on charcoal (Pd/C) at around 20 Kg pressure of hydrogen at a temperature of about 60° C. in methanolic-HCl; 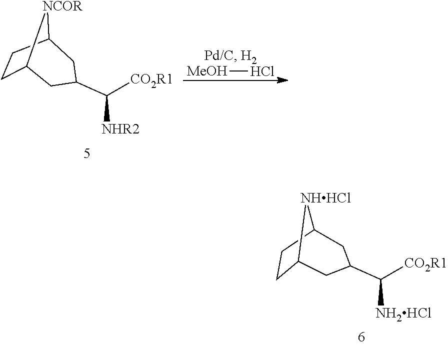
Wherein, R is selected from substituted or unsubstituted phenyl, C1 to C4 alkyloxy, substituted or unsubstituted benzyloxy; R1 is selected from C1 to C4 alkyl or benzyl; and R2 is selected from (R/S)—C*H(Ph)CH₂OH, (R/S)—C*H(Ph)CH₃ wherein, asterisk denotes point of attachment;
(v) reaction of compound of formula 6 with benzyl chloroformate in the presence of aqueous saturated sodium bicarbonate solution in aqueous 1,4-dioxane at around 0° C. to give compound of formula 7; 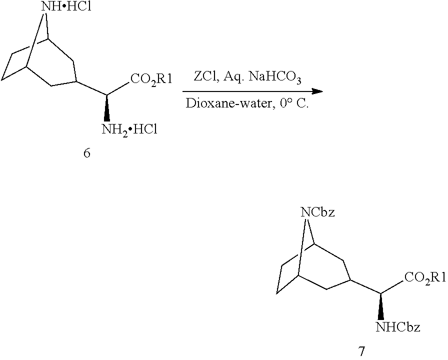
Wherein, R1 is selected from C1 to C4 alkyl or benzyl;
(vi) conversion of compound of formula 7 to compound of formula 8 using di-tert-butyl dicarbonate and 4-dimethylaminopyridine in an aprotic organic solvent such as acetonitrile at room temperature; 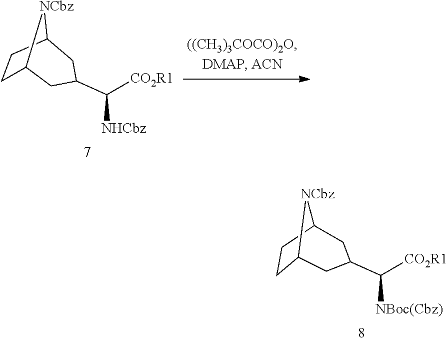
wherein, R1 is selected from C1 to C4 alkyl or benzyl;
(vii) hydrogenolysis of compound of formula 8 over palladium supported on charcoal (Pd/C) at around 5 Kg pressure of hydrogen at room temperature to give compound of formula n; 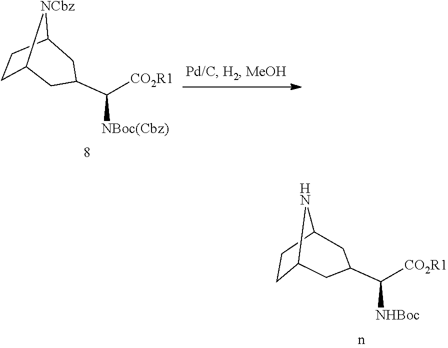
wherein, R1 is selected from C1 to C4 alkyl or benzyl;
such that,
at each step the product is optionally isolated and purified by techniques such as crystallization, column chromatography or distillation.

2. A process for the synthesis of methyl (2S)-2-(tert-butoxycarbonyl)-amino-2-[-8-azabicyclo[3.2.1]oct-3-yl]-exo-acetate comprising of hydrogenolysis of methyl (2S)-2-((tert-butoxycarbonyl)-[benzyloxy carbonyl]-amino)-2-[8-(benzyloxy carbonyl)-8-azabicyclo[3.2.1]oct-3-yl]-exo-acetate in presence of palladium supported on charcoal at around 5 Kg pressure of hydrogen at room temperature followed by isolation of the product formed.

3. The process as claimed in claim 2, wherein methyl (2S)-2-((tert-butoxycarbonyl)-[benzyloxy carbonyl]-amino)-2-[8-(benzyloxy carbonyl)-8-azabicyclo[3.2.1]oct-3-yl]-exo-acetate is synthesized by reaction of methyl (2S)-2-(benzyloxycarbonyl)-amino-2-[-8-[benzyloxy carbonyl]-8-azabicyclo[3.2.1]oct-3-yl]-exo-acetate with di-tert-butyl dicarbonate and 4-dimethylamino pyridine in an aprotic organic solvent such as acetonitrile at room temperature.

4. The process as claimed in claim 3, wherein methyl (2S)-2-(benzyloxycarbonyl)-amino-2-[-8-[benzyloxy carbonyl]-8-azabicyclo-[3.2.1]oct-3-yl]-exo-acetate is prepared by reaction of methyl (2S)-2-amino-2-[8-azabicyclo-[3.2.1]oct-3-yl]-exo-acetate hydrochloride with benzyloxy carbonyl chloride in the presence of aqueous sodium bicarbonate solution in aqueous 1,4-dioxane at around 0° C.

5. The process as claimed in claim 4 wherein, methyl (2S)-2-amino-2-[8-azabicyclo-[3.2.1]oct-3-yl]-exo-acetate hydrochloride is prepared by hydrogenolysis of 1-(2-hydroxy-1-(1R)-phenylethyl amino)-1-(8-(benzyloxy carbonyl)-8-aza-bicyclo[3.2.1]-oct-3-yl)-exo-methane-1-(1S)-carboxylic acid methyl ester hydrochloride over palladium supported on charcoal at around 20 Kg pressure of hydrogen at a temperature of about 60° C. in an organic solvent such as methanolic hydrochloric acid.

6. The process as claimed in claim 5 wherein, 1-(2-hydroxy-1-(1R)-phenylethyl amino)-1-(8-(benzyloxy carbonyl)-8-aza-bicyclo[3.2.1]-oct-3-yl)-exo-methane-1-(1S)-carboxylic acid methyl ester hydrochloride is prepared by a process comprising hydrolysis of the nitrile group in 1-(2-hydroxy-1-(1R)-phenylethyl amino)-1-(8-(benzyloxy carbonyl)-8-aza-bicyclo[3.2.1]-oct-3-yl)-exo-methane-1-(1S)-carbonitrile using methanolic-HCl a room temperature.

7. The process as claimed in claim 6 wherein, 1-(2-hydroxy-1-(1R)-phenylethyl amino)-1-(8-(benzyloxy carbonyl)-8-aza-bicyclo[3.2.1]-oct-3-yl)-exo-methane-1-(1S)-carbonitrile is prepared by a process comprising of reaction of benzyl 3-exo-formyl-8-azabicyclo[3.2.1]octane-8-carboxylate with R-(−)-2-Phenyl glycinol and trimethylsilylcyanide in an organic solvent selected from dichloromethane, methanol, chloroform, 1,2-ethylenedichloride, hexane, tetrahydrofuran, methyltetrahydrofuran, DME or mixtures thereof under standard Strecker reaction conditions in the presence of acetic acid, or Lewis acid at a temperature between −40 and 25° C.

8. The process as claimed in claim 7 wherein, benzyl 3-exo-formyl-8-azabicyclo[3.2.1]octane-8-carboxylate is prepared by a process comprising:
(a) reaction of 8-methyl-8-azabicyclo[3.2.1]octan-3-one with benzyl chloroformate in presence of an inorganic base such as K₂CO₃ in a organic solvent such as toluene at an ambient temperature to obtain benzyl 3-oxo-8-azabicyclo[3.2.1]octane-8-carboxylate followed by optional isolation and purification of the product;
(b) reaction of benzyl 3-oxo-8-azabicyclo[3.2.1]octane-8-carboxylate with glide obtained by reaction of sodium hydride and trimethylsulfonium iodide in an organic solvent such as dimethylformamide at room temperature to obtain benzyl 8H-spiro[8-azabicyclo[3.2.1]octane-3,2′-oxirane]-8-carboxylate followed by optional isolation and purification of the product;
(c) reaction of benzyl 8H-spiro[8-azabicyclo[3.2.1]octane-3,2′-oxirane]-8-carboxylate with BF₃-etherate in an organic solvent such as dichloromethane at room temperature to obtain benzyl 3-exo-formyl-8-azabicyclo[3.2.1]octane-8-carboxylate followed by optional isolation and purification of the product.

9. The process as claimed in claim 1 further comprises conversion of compound of formula n 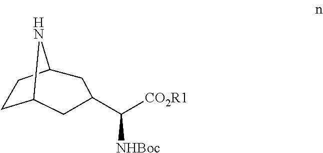
wherein, R1 is selected from C1 to C4 alkyl or benzyl; to compound of formula o 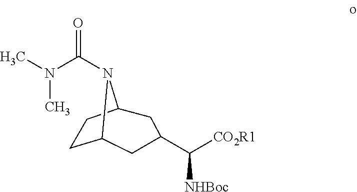
wherein, R1 is selected from C1 to C4 alkyl or benzyl; comprising conversion of compound of formula n to compound of formula o by a carbonyl insertion reaction between intermediate o and N,N dimethyl amine using triphosgene or di-tert-butyl dicarbonate or 1,1-carbonyl bis imidazole and an organic base such as diisopropyl ethyl amine, triethyl amine or an inorganic base such as cesium carbonate in an appropriate organic solvent such as dimethylformamide, dimethylacetamide or any water miscible cyclic ether.

10. The process as claimed in claim 1 further comprises conversion of compound of formula n 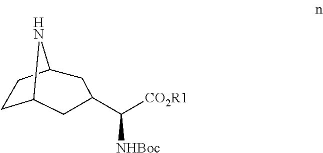
wherein, R1 is selected from C1 to C4 alkyl or benzyl; to compound of formula o 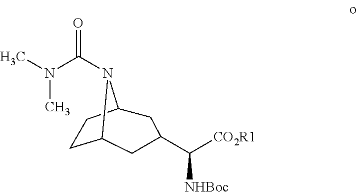
wherein, R1 is selected from C1 to C4 alkyl or benzyl; comprising treatment of compound of formula n with N,N-dimethyl carbamoyl chloride in presence of a suitable base such as triethylamine, N,N-diisopropyl ethyl amine in an organic solvent.

11-16. (canceled)

17. A process for the synthesis of compound of formula o 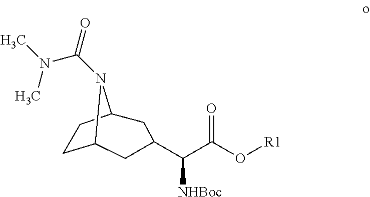
wherein, R1 is selected from methyl and ethyl; comprising:
i) conversion of 1-(2-hydroxy-1-(1R)-phenylethyl amino)-1-(8-(benzyloxy carbonyl)-8-aza-bicyclo[3.2.1]-oct-3-yl)-exo-methane-1-(1S)-carboxylic acid methyl ester hydrochloride salt (compound 5) to 1-(2-hydroxy-1-(1R)-phenylethyl amino)-1-(8-(dimethyl carbamoyl)-8-aza-bicyclo[3.2.1]-oct-3-yl)-exo-methane-1-(1S)-carboxylic acid methyl ester (compound 10) by selective hydrogenation of benzyloxy carbonyl group in 5 to give intermediate 9 using Pd/C catalyst at an elevated pressure of hydrogen at room temperature followed by regio-selective introduction of N,N-dimethyl carbamoyl group on the bridge head nitrogen in the presence of an organic base such as diisopropylethylamine or triethylamine or using an inorganic bases such as cesium carbonate in an appropriate organic solvents such as dimethyl formamide or dimethyl acetamide or in any water miscible cyclic ether; 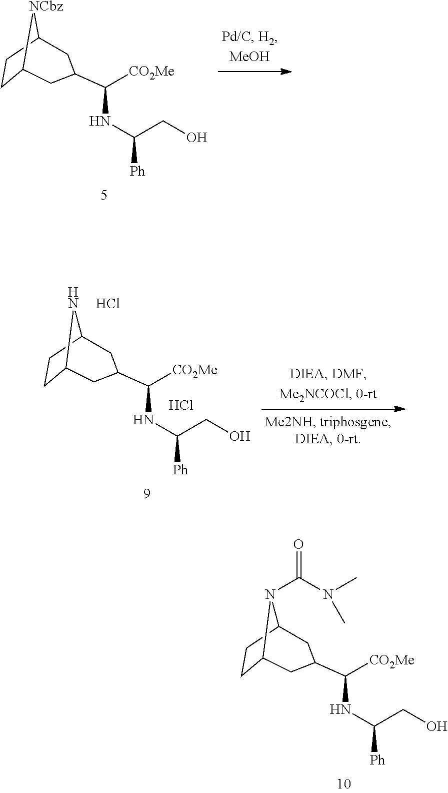
or
conversion of 1-(2-hydroxy-1-(1R)-phenylethyl amino)-1-(8-(benzyloxy carbonyl)-8-aza-bicyclo[3.2.1]-oct-3-yl)-exo-methane-1-(1S)-carbonitrile hydrochloride salt (compound 4) to 1-(2-hydroxy-1-(1R)-phenylethyl amino)-1-(8-(dimethyl carbamoyl)-8-aza-bicyclo[3.2.1]-oct-3-yl)-exo-methane-1-(1S)-carboxylic acid methyl ester (compound 10) by treating a solution of compound 4 with dry hydrogen chloride gas for extended period of time at room temperature to give (S)-(8-Aza-bicyclo[3.2.1]oct-3-yl)-((R)-2-hydroxy-1-phenyl-ethylamino)-acetic acid methyl ester 9 followed by regio-selective introduction of N,N-dimethyl carbamoyl group using N,N-dimethyl carbamoyl chloride or N,N-dimethylamine and triphosgene on the bridge head nitrogen in the presence of an organic base such as diisopropylethylamine or triethylamine or using an inorganic bases such as cesium carbonate in an appropriate organic solvents such as dimethyl formamide or dimethyl acetamide or in any water miscible cyclic ether at temperature ranging between 0 to 35°.
ii) conversion of 1-(2-hydroxy-1-(1R)-phenylethyl amino)-1-(8-(dimethyl carbamoyl)-8-aza-bicyclo[3.2.1]-oct-3-yl)-exo-methane-1-(1S)-carboxylic acid methyl ester (compound 10) to methyl (2S)-2-(tert-butoxycarbonyl)-amino-2-[-8-[dimethylcarbamoyl]-8-azabicyclo[3.2.1]-oct-3-yl]-exo-acetate (compound o) in two steps comprising of removal of the phenyl ethanol group in 10 by hydrogenation in presence of Pd/C catalyst and acetic acid in an organic solvent such as methanol at around 20 Kg hydrogen pressure to form compound of formula II, followed by protection of the free amine by treatment with di-tert-butyl dicarbonate ([(CH₃)₃COCO]₂O) in dichloromethane; 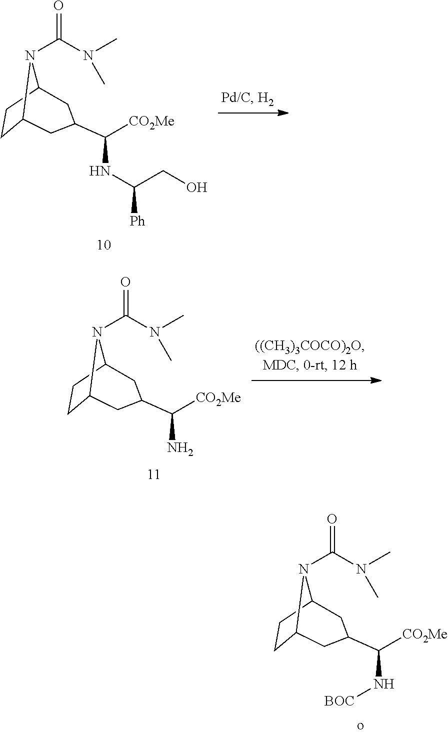
such that at each step the intermediates were optionally isolated and purified with suitable process.

18. A process for the synthesis of methyl (2S)-2-(tert-butoxycarbonyl)-amino-2-[-8-[dimethylcarbamoyl]-8-azabicyclo[3.2.1]-oct-3-yl]-exo-acetate comprising of hydrogenolysis of 1-(2-hydroxy-1-(1R)-phenylethyl amino)-1-(8-(dimethyl carbamoyl)-8-aza-bicyclo[3.2.1]-oct-3-yl)-exo-methane-1-(1S)-carboxylic acid methyl ester in presence of palladium supported on charcoal at around 5 Kg pressure of hydrogen at room temperature followed by treatment with di-tert-butyl dicarbonate (RCH₃)₃COCO]₂O) in dichloromethane at an temperature ranging between 0 to 35° C.

19. The process as claimed in claim 18, wherein 1-(2-hydroxy-1-(1R)-phenylethyl amino)-1-(8-(dimethyl carbamoyl)-8-aza-bicyclo[3.2.1]-oct-3-yl)-exo-methane-1-(1S)-carboxylic acid methyl ester is synthesized by a process comprising selective hydrogenation of 1-(2-hydroxy-1-(1R)-phenylethyl amino)-1-(8-(benzyloxy carbonyl)-8-aza-bicyclo[3.2.1]-oct-3-yl)-exo-methane-1-(1S)-carboxylic acid methyl ester hydrochloride salt using palladium supported on carbon at around 5 Kg pressure of hydrogen at room temperature followed by treatment with N,N-dimethylcarbamoyl chloride or dimethylamine and triphosgene in presence of a suitable base such as N,N-diisopropyl ethylamine or triethylamine, or using an inorganic base such as cesium carbonate in an appropriate organic solvents such as dimethyl formamide or dimethyl acetamide or in any water miscible cyclic ether at temperature ranging between 0 to 35.

20. The process as claimed in claim 19, wherein 1-(2-hydroxy-1-(1R)-phenylethyl amino)-1-(8-(benzyloxy carbonyl)-8-aza-bicyclo[3.2.1]-oct-3-yl)-exo-methane-1-(1S)-carboxylic acid methyl ester hydrochloride salt is synthesized by a process comprising selective hydrogenation of 1-(2-hydroxy-1-(1R)-phenylethyl amino)-1-(8-(benzyloxy carbonyl)-8-aza-bicyclo[3.2.1]-oct-3-yl)-exo-methane-1-(1S)-carboxylic acid methyl ester hydrochloride salt using palladium supported on carbon at around 5 Kg pressure of hydrogen at room temperature followed by treatment with dimethylamine and triphosgene in presence of a suitable base such as N,N-diisopropyl ethylamine or triethylamine, or using an inorganic base such as cesium carbonate in an appropriate organic solvents such as dimethyl formamide or dimethyl acetamide or in any water miscible cyclic ether at temperature ranging between 0 to 35.

21. The process as claimed in claim 20 wherein, 1-(2-hydroxy-1-(1R)-phenylethyl amino)-1-(8-(benzyloxy carbonyl)-8-aza-bicyclo[3.2.1]-oct-3-yl)-exo-methane-1-(1S)-carboxylic acid methyl ester hydrochloride is prepared by a process comprising hydrolysis of the nitrile group in 1-(2-hydroxy-1-(1R)-phenylethyl amino)-1-(8-(benzyloxy carbonyl)-8-aza-bicyclo[3.2.1]-oct-3-yl)-exo-methane-1-(1S)-carbonitrile using methanolic-HCl a room temperature.

22. The process as claimed in claim 21 wherein, 1-(2-hydroxy-1-(1R)-phenylethyl amino)-1-(8-(benzyloxy carbonyl)-8-aza-bicyclo[3.2.1]-oct-3-yl)-exo-methane-1-(1S)-carbonitrile is prepared by a process comprising of reaction of benzyl 3-exo-formyl-8-azabicyclo[3.2.1]octane-8-carboxylate with R-(−)-2-Phenyl glycinol and trimethylsilylcyanide in an organic solvent selected from dichloromethane, methanol, chloroform, 1,2-ethylenedichloride, hexane, tetrahydrofuran, methyltetrahydrofuran, DME or mixtures thereof under standard Strecker reaction conditions in the presence of acetic acid, or Lewis acid at a temperature between −40 and 25° C.

23. The process as claimed in claim 22 wherein, benzyl 3-exo-formyl-8-azabicyclo[3.2.1]octane-8-carboxylate is prepared by a process comprising:
(a) reaction of 8-methyl-8-azabicyclo[3.2.1]octan-3-one with benzyl chloroformate in presence of an inorganic base such as K₂CO₃ in a organic solvent such as toluene at an ambient temperature to obtain benzyl 3-oxo-8-azabicyclo[3.2.1]octane-8-carboxylate followed by optional isolation and purification of the product;
(b) reaction of benzyl 3-oxo-8-azabicyclo[3.2.1]octane-8-carboxylate with ylide obtained by reaction of sodium hydride and trimethylsulfonium iodide in an organic solvent such as dimethylformamide at room temperature to obtain benzyl 8H-spiro[8-azabicyclo[3.2.1]octane-3,2′-oxirane]-8-carboxylate followed by optional isolation and purification of the product;
(c) reaction of benzyl 8H-spiro[8-azabicyclo[3.2.1]octane-3,2′-oxirane]-8-carboxylate with BF₃-etherate in an organic solvent such as dichloromethane at room temperature to obtain benzyl 3-exo-formyl-8-azabicyclo[3.2.1]octane-8-carboxylate followed by optional isolation and purification of the product.

24-25. (canceled)

26. A process for the synthesis of compound of formula n 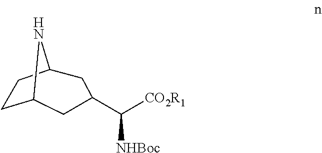
wherein, R1 is selected from C1 to C4 alkyl or benzyl; comprising:
i. conversion of compound of formula 1 to compound of formula 12 by the condensation of compound of formula 1 with an active methylene compounds such as methyl or ethyl isocyanoacetate in the presence of sodium hydride (NaH) in an aprotic solvent such as tetrahydrofuran (THF) or in 1,4-dioxane or any water miscible cyclic ether at about a temperature between 0 and 10° C.; 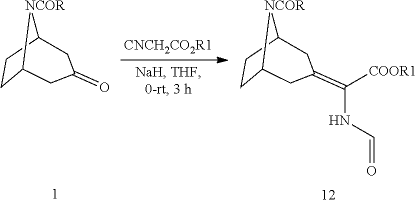
Wherein, R is selected from substituted or unsubstituted phenyl, C1 to C4 alkyloxy, substituted or unsubstituted benzyloxy; and R1 is selected from C1 to C4 alkyl or benzyl;
ii. conversion of compound of formula 12 to compound of formula 13 with exo configuration by treatment with methanolic-HCl at around 0° C.; 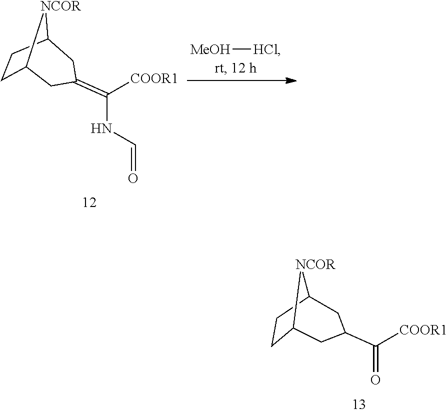
Wherein, R is selected from substituted or unsubstituted phenyl, C1 to C4 alkyloxy, substituted or unsubstituted benzyloxy; and R1 is selected from C1 to C4 alkyl or benzyl;
iii. conversion of compound of formula 13 to compound of formula 15 by first treating compound 13 with either (S)-(−)-2-methyl-2-propane sulfinamide or with (R)-(+)-2-methyl-2-propane sulfinamide in the presence of titanium(IV) ethoxide at reflux temperature followed by further reduction of the intermediate ketimine using a suitable reducing agent such as sodium borohydride or sodium triacetoxy borohydride or any modified borohydrides derived from a combination of sodium borohydride with chiral acid such as camphoric acid or tartaric acid or with a achiral acid such as succinic or phthalic acid at room temperature; 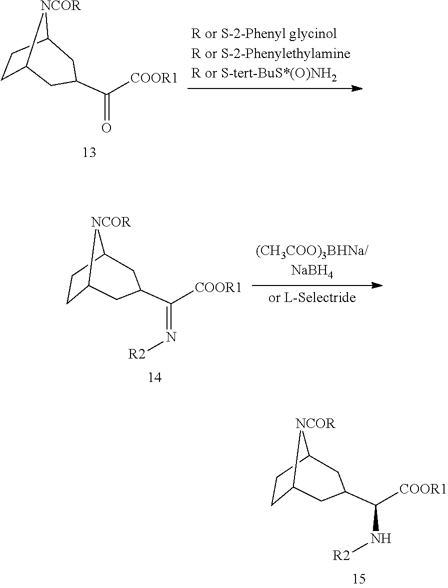
Wherein, R is selected from substituted or unsubstituted phenyl, C1 to C4 alkyloxy, substituted or unsubstituted benzyloxy; R1 is selected from C1 to C4 alkyl or benzyl, and R2 is (R/S) tert-C₄H₉—S*(O), wherein, asterisk denotes point of attachment;
iv. conversion of compound of formula 15 to compound m first by treatment with alcoholic-HCl, followed by treatment with di-tert-butyl dicarbonate, further hydrogenolysis of compound of formula m over palladium supported on charcoal (Pd/C) to give compound of formula n; 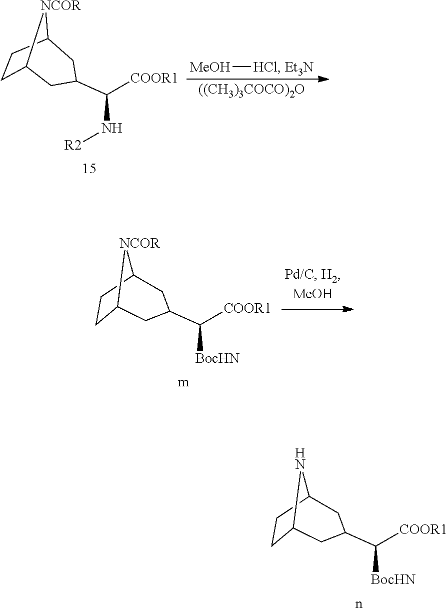
Wherein, R is selected from substituted or unsubstituted phenyl, C1 to C4 alkyloxy, substituted or unsubstituted benzyloxy; R1 is selected from C1 to C4 alkyl or benzyl, and R2 is (R/S) tert-C₄H₉—S*(O), wherein, asterisk denotes point of attachment;
Such that at each step the intermediates were optionally isolated and purified with suitable process.

27. A process for the synthesis of methyl (2S)-2-(tert-butoxycarbonyl)-amino-2-[-8-azabicyclo[3.2.1]oct-3-yl]-exo-acetate comprising conversion of 1-(R or S-tert-butylsulfinylamino)-1-(8-(benzyloxy carbonyl)-8-aza-bicyclo[3.2.1]-oct-3-yl)-exo-methane-1-(1S)-carboxylic acid methyl ester (compound 15) to methyl (2S)-2-((tert-butoxycarbonyl)-amino)-2-[8-(benzyloxy carbonyl)-8-azabicyclo[3.2.1]oct-3-yl]-exo-acetate (compound m) first by treatment with methanolic-HCl, followed by treatment with di-tert-butyl dicarbonate. Further hydrogenolysis of compound of formula m over palladium supported on charcoal (Pd/C) to give the product.

28. The process as claimed in claim 27 wherein, 1-(R or S-tert-butylsulfinylamino)-1-(8-(benzyloxy carbonyl)-8-aza-bicyclo[3.2.1]-oct-3-yl)-exo-methane-1-(1S)-carboxylic acid ethyl ester with methanolic-HCl is prepared by conversion of benzyl exo-3-[ethoxy or methoxy (oxo)acetyl]-8-azabicyclo[3.2.1]octane-8-carboxylate (compound 13) to 1-(R or S-tert-butylsulfinylamino)-1-(8-(benzyloxy carbonyl)-8-aza-bicyclo[3.2.1]-oct-3-yl)-exo-methane-1-(1S)-carboxylic acid ethyl ester (compound 15) by first treating compound 13 with either (R)-(+)-2-methyl-2-propane sulfinamide or with (S)-(−)-2-methyl-2-propane sulfinamide in the presence of titanium(IV) ethoxide and followed by further reduction of the intermediate ketimine 14 using a suitable reducing agent such as sodium borohydride or sodium triacetoxy borohydride or modified borohydrides derived from a combination of sodium borohydride with chiral acid such as camphoric acid or tartaric acid or with a achiral acid such as succinic or phthalic acid.

29. The process as claimed in claim 28 wherein, benzyl exo-3-[ethoxy or methoxy (oxo)acetyl]-8-azabicyclo[3.2.1]octane-8-carboxylate is prepared by a process comprising treatment of benzyl 3-(1-formamido-2-ethoxy-2-(oxo)ethylidene)-8-azabicyclo[3.2.1]-octane-8-carboxylate (compound 12) with methanolic-HCl.

30. The process as claimed in claim 29 wherein, benzyl 3-(1-formamido-2-ethoxy-2-(oxo)ethylidene)-8-azabicyclo[3.2.1]-octane-8-carboxylate is prepared by a process comprising condensation of benzyl 3-oxo-8-azabicyclo[3.2.1]octane-8-carboxylate (compound 1) to with an active methylene compounds such as methyl or ethyl isocyanoacetate.

31-34. (canceled)

35. A compound selected from
benzyl 3-exo-formyl-8-azabicyclo[3.2.1]octane-8-carboxylate;
1-(2-hydroxy-1-(1R)-phenylethyl amino)-1-(8-(benzyloxy carbonyl)-8-aza-bicyclo[3.2.1]-oct-3-yl)-exo-methane-1-(1S)-carbonitrile and salts thereof;
1-(2-hydroxy-1-(1R)-phenylethyl amino)-1-(8-(benzyloxy carbonyl)-8-aza-bicyclo[3.2.1]-oct-3-yl)-exo-methane-1-(1S)-carboxylic acid methyl ester and salts thereof;
methyl (2S)-2-amino-2-[8-azabicyclo[3.2.1]oct-3-yl]-exo-acetate and salts thereof;
methyl (2S)-2-(benzyloxycarbonyl)-amino-2-[-8-[benzyloxy carbonyl]-8-azabicyclo[3.2.1]oct-3-yl]-exo-acetate;
benzyl methyl (2S)-2-((tert-butoxycarbonyl)-[benzyloxy carbonyl]-amino)-2-[8-(benzyloxy carbonyl)-8-azabicyclo[3.2.1]oct-3-yl]-exo-acetate;
1-(2-hydroxy-1-(1R)-phenylethyl amino)-1-(8-(dimethyl carbamoyl)-8-aza-bicyclo[3.2.1]-oct-3-yl)-exo-methane-1-(1S)-carboxylic acid methyl ester;
(S)-Amino-(8-dimethylcarbamoyl-8-aza-bicyclo[3.2.1]oct-3-yl)-acetic acid methyl ester;
benzyl 3-(1-formamido-2-ethoxy-2-(oxo)ethylidene)-8-azabicyclo[3.2.1]-octane-8-carboxylate;
benzyl exo-3-[ethoxy or methoxy (oxo)acetyl]-8-azabicyclo[3.2.1]octane-8-carboxylate;
benzyl 3-[(1E/Z)-N-((R or S)-tert-butylsulfinyl)-2-ethoxy-2-oxoethanimidoyl]-8-azabicyclo[3.2.1]octane-8-carboxylate; and
1-((R or S)-tert-butylsulfinylamino)-1-(8-(benzyloxy carbonyl)-8-aza-bicyclo[3.2.1]-oct-3-yl)-exo-methane-1-(1S)-carboxylic acid ethyl ester.
